SEQUENCE LISTING (71 sequences)

SEQ ID NO: 1 - DNA, 83 bp, Artificial Sequence
  Synthetic Oligonucleotide
  misc_feature (30)..(41): n is a, c, g, or t
aatgatacggcgaccaccgagatctacacnnnnnnnnnnnncctgcgaggcccgcatctttcacaaattttgtaatccagagg

SEQ ID NO: 2 - DNA, 83 bp, Artificial Sequence
  Synthetic Oligonucleotide
  misc_feature (30)..(41): n is a, c, g, or t
aatgatacggcgaccaccgagatctacacnnnnnnnnnnnngaggcgaggcccgcatctttcacaaattttgtaatccagagg

SEQ ID NO: 3 - DNA, 83 bp, Artificial Sequence
  Synthetic Oligonucleotide
  misc_feature (30)..(41): n is a, c, g, or t
aatgatacggcgaccaccgagatctacacnnnnnnnnnnnnttagcgaggcccgcatctttcacaaattttgtaatccagagg

SEQ ID NO: 4 - DNA, 83 bp, Artificial Sequence
  Synthetic Oligonucleotide
  misc_feature (30)..(41): n is a, c, g, or t
aatgatacggcgaccaccgagatctacacnnnnnnnnnnnnagcgcgaggcccgcatctttcacaaattttgtaatccagagg

SEQ ID NO: 5 - DNA, 83 bp, Artificial Sequence
  Synthetic Oligonucleotide
  misc_feature (30)..(41): n is a, c, g, or t
aatgatacggcgaccaccgagatctacacnnnnnnnnnnnnaatgcgaggcccgcatctttcacaaattttgtaatccagagg

SEQ ID NO: 6 - DNA, 83 bp, Artificial Sequence
  Synthetic Oligonucleotide
  misc_feature (30)..(41): n is a, c, g, or t
aatgatacggcgaccaccgagatctacacnnnnnnnnnnnncaagcgaggcccgcatctttcacaaattttgtaatccagagg

SEQ ID NO: 7 - DNA, 83 bp, Artificial Sequence
  Synthetic Oligonucleotide
  misc_feature (30)..(38): n is a, c, g, or t
aatgatacggcgaccaccgagatctacacnnnnnnnnnagtgtcgcgaggcccgcatctttcacaaattttgtaatccagagg

SEQ ID NO: 8 - DNA, 83 bp, Artificial Sequence
  Synthetic Oligonucleotide
  misc_feature (30)..(38): n is a, c, g, or t
aatgatacggcgaccaccgagatctacacnnnnnnnnntatccggcgaggcccgcatctttcacaaattttgtaatccagagg

SEQ ID NO: 9 - DNA, 83 bp, Artificial Sequence
  Synthetic Oligonucleotide
  misc_feature (30)..(38): n is a, c, g, or t
aatgatacggcgaccaccgagatctacacnnnnnnnnncatttggcgaggcccgcatctttcacaaattttgtaatccagagg

SEQ ID NO: 10 - DNA, 83 bp, Artificial Sequence
  Synthetic Oligonucleotide
  misc_feature (30)..(38): n is a, c, g, or t
aatgatacggcgaccaccgagatctacacnnnnnnnnnatgctagcgaggcccgcatctttcacaaattttgtaatccagagg

SEQ ID NO: 11 - DNA, 83 bp, Artificial Sequence
  Synthetic Oligonucleotide
  misc_feature (30)..(38): n is a, c, g, or t
aatgatacggcgaccaccgagatctacacnnnnnnnnnccgtgggcgaggcccgcatctttcacaaattttgtaatccagagg

SEQ ID NO: 12 - DNA, 83 bp, Artificial Sequence
  Synthetic Oligonucleotide
  misc_feature (30)..(38): n is a, c, g, or t
aatgatacggcgaccaccgagatctacacnnnnnnnnnatgagtgcgaggcccgcatctttcacaaattttgtaatccagagg

SEQ ID NO: 13 - DNA, 83 bp, Artificial Sequence
  Synthetic Oligonucleotide
  misc_feature (30)..(38): n is a, c, g, or t
aatgatacggcgaccaccgagatctacacnnnnnnnnncgagcagcgaggcccgcatctttcacaaattttgtaatccagagg

SEQ ID NO: 14 - DNA, 83 bp, Artificial Sequence
  Synthetic Oligonucleotide
  misc_feature (30)..(38): n is a, c, g, or t
aatgatacggcgaccaccgagatctacacnnnnnnnnncgcggcgcgaggcccgcatctttcacaaattttgtaatccagagg

SEQ ID NO: 15 - DNA, 83 bp, Artificial Sequence
  Synthetic Oligonucleotide
  misc_feature (30)..(38): n is a, c, g, or t
aatgatacggcgaccaccgagatctacacnnnnnnnnnacttatgcgaggcccgcatctttcacaaattttgtaatccagagg

SEQ ID NO: 16 - DNA, 83 bp, Artificial Sequence
  Synthetic Oligonucleotide
  misc_feature (30)..(38): n is a, c, g, or t
aatgatacggcgaccaccgagatctacacnnnnnnnnntgcatggcgaggcccgcatctttcacaaattttgtaatccagagg

SEQ ID NO: 17 - DNA, 83 bp, Artificial Sequence
  Synthetic Oligonucleotide
  misc_feature (30)..(38): n is a, c, g, or t
aatgatacggcgaccaccgagatctacacnnnnnnnnnagtagggcgaggcccgcatctttcacaaattttgtaatccagagg

SEQ ID NO: 18 - DNA, 83 bp, Artificial Sequence
  Synthetic Oligonucleotide
  misc_feature (30)..(38): n is a, c, g, or t
aatgatacggcgaccaccgagatctacacnnnnnnnnngttgacgcgaggcccgcatctttcacaaattttgtaatccagagg

SEQ ID NO: 19 - DNA, 83 bp, Artificial Sequence
  Synthetic Oligonucleotide
  misc_feature (30)..(38): n is a, c, g, or t
aatgatacggcgaccaccgagatctacacnnnnnnnnntatcacgcgaggcccgcatctttcacaaattttgtaatccagagg

SEQ ID NO: 20 - DNA, 83 bp, Artificial Sequence
  Synthetic Oligonucleotide
  misc_feature (30)..(38): n is a, c, g, or t
aatgatacggcgaccaccgagatctacacnnnnnnnnnccctaggcgaggcccgcatctttcacaaattttgtaatccagagg

SEQ ID NO: 21 - DNA, 83 bp, Artificial Sequence
  Synthetic Oligonucleotide
  misc_feature (30)..(38): n is a, c, g, or t
aatgatacggcgaccaccgagatctacacnnnnnnnnngcccgtgcgaggcccgcatctttcacaaattttgtaatccagagg

SEQ ID NO: 22 - DNA, 83 bp, Artificial Sequence
  Synthetic Oligonucleotide
  misc_feature (30)..(38): n is a, c, g, or t
aatgatacggcgaccaccgagatctacacnnnnnnnnnttcccggcgaggcccgcatctttcacaaattttgtaatccagagg

SEQ ID NO: 23 - DNA, 83 bp, Artificial Sequence
  Synthetic Oligonucleotide
  misc_feature (30)..(38): n is a, c, g, or t
aatgatacggcgaccaccgagatctacacnnnnnnnnncatatagcgaggcccgcatctttcacaaattttgtaatccagagg

SEQ ID NO: 24 - DNA, 83 bp, Artificial Sequence
  Synthetic Oligonucleotide
  misc_feature (30)..(38): n is a, c, g, or t
aatgatacggcgaccaccgagatctacacnnnnnnnnnaacgccgcgaggcccgcatctttcacaaattttgtaatccagagg

SEQ ID NO: 25 - DNA, 83 bp, Artificial Sequence
  Synthetic Oligonucleotide
  misc_feature (30)..(38): n is a, c, g, or t
aatgatacggcgaccaccgagatctacacnnnnnnnnnaggttggcgaggcccgcatctttcacaaattttgtaatccagagg

SEQ ID NO: 26 - DNA, 83 bp, Artificial Sequence
  Synthetic Oligonucleotide
  misc_feature (30)..(38): n is a, c, g, or t
aatgatacggcgaccaccgagatctacacnnnnnnnnntcaatagcgaggcccgcatctttcacaaattttgtaatccagagg

SEQ ID NO: 27 - DNA, 39 bp, Artificial Sequence
  Synthetic Oligonucleotide
gcgaggcccgcatctttcacaaattttgtaatccagagg

SEQ ID NO: 28 - DNA, 39 bp, Artificial Sequence
  Synthetic Oligonucleotide
cctctggattacaaaatttgtgaaagatgcgggcctcgc

SEQ ID NO: 29 - DNA, 66 bp, Artificial Sequence
  Synthetic Oligonucleotide
caagcagaagacggcatacgagatactggtcaaagttactatcgaaatgccctgagtccaccccgg

SEQ ID NO: 30 - DNA, 66 bp, Artificial Sequence
  Synthetic Oligonucleotide
caagcagaagacggcatacgagatgtgttcgtaagttactatcgaaatgccctgagtccaccccgg

SEQ ID NO: 31 - DNA, 66 bp, Artificial Sequence
  Synthetic Oligonucleotide
caagcagaagacggcatacgagattaactgttaagttactatcgaaatgccctgagtccaccccgg

SEQ ID NO: 32 - DNA, 66 bp, Artificial Sequence
  Synthetic Oligonucleotide
caagcagaagacggcatacgagatgattggtgaagttactatcgaaatgccctgagtccaccccgg

SEQ ID NO: 33 - DNA, 66 bp, Artificial Sequence
  Synthetic Oligonucleotide
caagcagaagacggcatacgagatggagagagaagttactatcgaaatgccctgagtccaccccgg

SEQ ID NO: 34 - DNA, 66 bp, Artificial Sequence
  Synthetic Oligonucleotide
caagcagaagacggcatacgagattgagcgataagttactatcgaaatgccctgagtccaccccgg

SEQ ID NO: 35 - DNA, 66 bp, Artificial Sequence
  Synthetic Oligonucleotide
caagcagaagacggcatacgagatcctccgttaagttactatcgaaatgccctgagtccaccccgg

SEQ ID NO: 36 - DNA, 66 bp, Artificial Sequence
  Synthetic Oligonucleotide
caagcagaagacggcatacgagataacatattaagttactatcgaaatgccctgagtccaccccgg

SEQ ID NO: 37 - DNA, 66 bp, Artificial Sequence
  Synthetic Oligonucleotide
caagcagaagacggcatacgagatcttacgtaaagttactatcgaaatgccctgagtccaccccgg

SEQ ID NO: 38 - DNA, 66 bp, Artificial Sequence
  Synthetic Oligonucleotide
caagcagaagacggcatacgagattgacgtagaagttactatcgaaatgccctgagtccaccccgg

SEQ ID NO: 39 - DNA, 66 bp, Artificial Sequence
  Synthetic Oligonucleotide
caagcagaagacggcatacgagatctatgtataagttactatcgaaatgccctgagtccaccccgg

SEQ ID NO: 40 - DNA, 66 bp, Artificial Sequence
  Synthetic Oligonucleotide
caagcagaagacggcatacgagattttgcagaaagttactatcgaaatgccctgagtccaccccgg

SEQ ID NO: 41 - DNA, 66 bp, Artificial Sequence
  Synthetic Oligonucleotide
caagcagaagacggcatacgagatggtagcgaaagttactatcgaaatgccctgagtccaccccgg

SEQ ID NO: 42 - DNA, 66 bp, Artificial Sequence
  Synthetic Oligonucleotide
caagcagaagacggcatacgagatacgggtttaagttactatcgaaatgccctgagtccaccccgg

SEQ ID NO: 43 - DNA, 66 bp, Artificial Sequence
  Synthetic Oligonucleotide
caagcagaagacggcatacgagattaaacctcaagttactatcgaaatgccctgagtccaccccgg

SEQ ID NO: 44 - DNA, 66 bp, Artificial Sequence
  Synthetic Oligonucleotide
caagcagaagacggcatacgagatgagaactgaagttactatcgaaatgccctgagtccaccccgg

SEQ ID NO: 45 - DNA, 66 bp, Artificial Sequence
  Synthetic Oligonucleotide
caagcagaagacggcatacgagatggtttgataagttactatcgaaatgccctgagtccaccccgg

SEQ ID NO: 46 - DNA, 66 bp, Artificial Sequence
  Synthetic Oligonucleotide
caagcagaagacggcatacgagattagattataagttactatcgaaatgccctgagtccaccccgg

SEQ ID NO: 47 - DNA, 66 bp, Artificial Sequence
  Synthetic Oligonucleotide
caagcagaagacggcatacgagataaggttagaagttactatcgaaatgccctgagtccaccccgg

SEQ ID NO: 48 - DNA, 66 bp, Artificial Sequence
  Synthetic Oligonucleotide
caagcagaagacggcatacgagatccgaaaataagttactatcgaaatgccctgagtccaccccgg

SEQ ID NO: 49 - DNA, 34 bp, Artificial Sequence
  Synthetic Oligonucleotide
aagttactatcgaaatgccctgagtccaccccgg

SEQ ID NO: 50 - DNA, 34 bp, Artificial Sequence
  Synthetic Oligonucleotide
ccggggtggactcagggcatttcgatagtaactt

SEQ ID NO: 51 - DNA, 175 bp, Artificial Sequence
  Synthetic Oligonucleotide
agtacgcgttagattagattagattagattagattagattagaaaaattaatacgtacaccatcagggtacgtctcagacaccatcagggtctgtctggtacagcatcagcgtaccatatattttttccaatccaatccaatccaatccaatccaatccaaatagatcctaatca

SEQ ID NO: 52 - DNA, 286 bp, Artificial Sequence
  Synthetic Oligonucleotide
ttagattagattagattagattagattagattagaaaaattaatatacgtacaccatcagggtacgtcatatattttttccaatccaatccaatccaatccaatccaatccaatacgcgttagattagattagattagattagattagattagaaaaattaatacgtacaccatcagggtacgtctcagacaccatcagggtctgtctggtacagcatcagcgtaccatatattttttccaatccaatccaatccaatccaatccaatccaaatagatcctaatca

SEQ ID NO: 53 - DNA, 175 bp, Artificial Sequence
  Synthetic Oligonucleotide
agtacgcgttagattagattagattagattagattagattagaaaaattaatacgtacaccatcagggtacgtctcagacaccatcagggtctgtctggtacagcatcagcgtaccatatattttttctaatctaatctaatctaatctaatctaatctaaatagatcctaatca

SEQ ID NO: 54 - DNA, 285 bp, Artificial Sequence
  Synthetic Oligonucleotide
ttagattagattagattagattagattagattagaaaaattaatatacgtacaccatcagggtacgtcatatattttttctaatctaatctaatctaatctaatctaatctaaacgcgttagattagattagattagattagattagattagaaaaattaatacgtacaccatcagggtacgtctcagacaccatcagggtctgtctggtacagcatcagcgtaccatatattttttctaatctaatctaatctaatctaatctaatctaaatagatcctaatca

SEQ ID NO: 55 - DNA, 175 bp, Artificial Sequence
  Synthetic Oligonucleotide
agtacgcgttaaattatattaactaaattatagattaacaagaatattaaatacgtacaccatcagggtacgtctcagacaccatcagggtctgtctggtacagcatcagcgtacctatttaatattcttgttaatctataatttagttaatataatttaaatagatcctaatca

SEQ ID NO: 56 - DNA, 175 bp, Artificial Sequence
  Synthetic Oligonucleotide
agtacgcgattggttaatcccattggttaatcccattggttaatcccttaatacgtacaccatcagggtacgtctcagacaccatcagggtctgtctggtacagcatcagcgtaccatatatgggttaaactgatgggttaaactgatgggttaaactgatatagatcctaatca

SEQ ID NO: 57 - DNA, 175 bp, Artificial Sequence
  Synthetic Oligonucleotide
agtacgcgaaaaaaaaaaaaaaaaaaaaaaaaaaaaaaaaaaaaaaaaaaaaacgtacaccatcagggtacgtctcagacaccatcagggtctgtctggtacagcatcagcgtacctttttttttttttttttttttttttttttttttttttttttttttatagatcctaatca

SEQ ID NO: 58 - RNA, 10 nt, Artificial Sequence
  Synthetic Oligonucleotide
uagauuagau

SEQ ID NO: 59 - RNA, 10 nt, Artificial Sequence
  Synthetic Oligonucleotide
auccaaucca

SEQ ID NO: 60 - RNA, 10 nt, Artificial Sequence
  Synthetic Oligonucleotide
  misc_feature (2)..(2): n is inosine
  misc_feature (7)..(7): n is inosine
ungauungau

SEQ ID NO: 61 - RNA, 10 nt, Artificial Sequence
  Synthetic Oligonucleotide
  misc_feature (6)..(6): n is inosine
auccanucca

SEQ ID NO: 62 - RNA, 35 nt, Artificial Sequence
  Synthetic Oligonucleotide
uccaauccaauccaauccaauccaauccaauccaa

SEQ ID NO: 63 - RNA, 35 nt, Artificial Sequence
  Synthetic Oligonucleotide
uuagauuagauuagauuagauuagauuagauuaga

SEQ ID NO: 64 - RNA, 29 nt, Artificial Sequence
  Synthetic Oligonucleotide
ucuaaucuaaucuaaucuaaucuaaucua

SEQ ID NO: 65 - RNA, 29 nt, Artificial Sequence
  Synthetic Oligonucleotide
uagauuagauuagauuagauuagauuaga

SEQ ID NO: 66 - RNA, 33 nt, Artificial Sequence
  Synthetic Oligonucleotide
aaauuauauuaacuaaauuauagauuaacaaga

SEQ ID NO: 67 - RNA, 33 nt, Artificial Sequence
  Synthetic Oligonucleotide
ucuuguuaaucuauaauuuaguuaauauaauuu

SEQ ID NO: 68 - RNA, 36 nt, Artificial Sequence
  Synthetic Oligonucleotide
ucgguuaaucccauugguuaaucccauugguuaauc

SEQ ID NO: 69 - RNA, 37 nt, Artificial Sequence
  Synthetic Oligonucleotide
gguuaaacugauggguuaaacugauggguuaaaccga

SEQ ID NO: 70 - DNA, 34 bp, Artificial Sequence
  Synthetic Oligonucleotide
tttttttttttttttttttttttttttttttttt

SEQ ID NO: 71 - DNA, 34 bp, Artificial Sequence
  Synthetic Oligonucleotide
aaaaaaaaaaaaaaaaaaaaaaaaaaaaaaaaaa

(19) United States
(12) Patent Application Publication
(10) Pub. No.: US 2021/0348217 A1
(43) Pub. Date: Nov. 11, 2021

(54) RNA TICKERTAPE FOR RECORDING TRANSCRIPTIONAL HISTORIES OF CELLS

(71) Applicants: Massachusetts Institute of Technology, Cambridge, MA (US); BROAD INSTITUTE, INC, Cambridge, MA (US)

(72) Inventors: Samuel Gordon RODRIQUES, Cambridge, MA (US); Edward BOYDEN, Chestnut Hill, MA (US); Fei CHEN, Cambridge, MA (US); Linlin CHEN, Cambridge, MA (US); Sophia LIU, Roxbury, MA (US); Ellen ZHONG, Cambridge, MA (US); Joseph SCHERRER, Boston, MA (US)

(21) Appl. No.: 17/150,620

(22) PCT Filed: Jul. 16, 2019
(86) PCT No.: PCT/US19/41962

§ 371 Date: Jan. 15, 2021

Related U.S. Application Data
(60) Provisional application No. 62/698,490, filed on Jul. 16, 2018.

(51) Int. Cl. C12Q 1/6811; C12N 9/78; C07K 14/005

(52) U.S. Cl. CPC C12Q 1/6811; C12N 9/78; C12Y 305/04005; C12Y 305/04004; C07K 14/005

(57) ABSTRACT

The invention, in some aspects, includes systems, methods and components of molecular recorders that encode the timing of transcriptional activity into the sequence of RNA, which can then enable a sequencing-based readout of the internal dynamics of cells.

### RELATED APPLICATIONS

This application claims the benefit under 35 U.S.C. § 119(e) of U.S. Provisional application Ser. No. 62/698490 filed Jul. 16, 2018, the disclosure of which is incorporated by reference herein in its entirety.

### GOVERNMENT INTEREST

This invention was made with government support under grants 1DP5OD024583, 1R01MH114031 6937063, 1RM1HG008525 6932903, 2R01DA029639 6932279, 1R01MH103910 6928799, and 1DP1NS087724 6928706 from the National Institutes of Health and grant W911NF1510548 6933228 from the US Army Research Office. The government has certain rights in the invention.

### FIELD OF THE INVENTION

The invention, in some aspects, includes systems, methods and components of molecular recorders that encode the timing of transcriptional activity into the sequence of RNA, which can then enable a sequencing-based readout of the internal dynamics of cells.

### BACKGROUND OF THE INVENTION

Despite several new methods for recording cell-state information into sequences of DNA in living cells, no method has been available that can record the absolute timing of cellular events into nucleic acid form in eukaryotic cells. All molecular recorders to date use DNA as a substrate, and record the occurrence of cellular events into the sequence of the DNA (1-10). These methods have shown promise for positioning events within the cellular lineage: because the substrates are DNA based, newly created DNA molecules retain the edits of the parent molecule, so the locations of events within the lineage can be inferred by sequencing the DNA at an endpoint and reconstructing the phylogeny of the reporter. However, these methods are largely incapable of reporting on the timings of cellular events in absolute time. The only methods of molecular recording that have so far achieved absolute timing are incompatible with eukaryotic cell biology, and have resolutions on the order of days, which is too slow for tracking the dynamics of most cellular processes (5).

### SUMMARY OF THE INVENTION

According to an aspect of the invention, RNA-based molecular recording systems are provided. The systems include a reporter RNA (repRNA) and a predetermined enzyme, wherein an alteration in an original composition of the repRNA indicates one or more of (a) an age of the repRNA and (b) a response of the repRNA to an applied stimulus. In some embodiments, the repRNA comprises an editing array in the 3′ UTR of a target RNA, wherein (i) the editing array comprises one or more engineered binding sites; and (ii) the editing array comprises one or more selectively favored substrates of the predetermined enzyme. In certain embodiments, the target RNA is an endogenous RNA. In certain embodiments, the target RNA is an mRNA. In some embodiments, the editing array comprises at least one of an adenosine-rich editing array and a cytosine-rich editing array. In some embodiments, the predetermined enzyme is attached to a binding polypeptide capable of binding to the engineered binding site in the repRNA editing array. In some embodiments, the engineered binding site is an engineered MS2 binding site. In certain embodiments, the binding polypeptide is an MS2 Capsid protein (MCP) and is capable of binding the MS2 binding sites engineered into the repRNA editing array. In some embodiments, the editing array comprises an adenosine-rich editing array and the predetermined enzyme comprises an Adenosine Deaminase Acting on RNA (ADAR) enzyme. In certain embodiments, the editing array comprises a cytosine-rich editing array and the predetermined enzyme comprises a Cytosine Deaminase Acting on RNA (CDAR) enzyme. In some embodiments, the ADAR or CDAR enzyme is a modified ADAR2 enzyme or modified CDAR enzyme, respectively. In some embodiments, the modified ADAR enzyme is a modified human ADAR2 enzyme or a modified mouse ADAR2 enzyme. In certain embodiments, the predetermined enzyme is ADAR or CDAR and the molecular recording system comprises an MCP-ADAR fusion protein or an MCP-CDAR fusion protein, respectively. In some embodiments, the predetermined enzyme is ADAR E488QT490A, and the molecular recording system comprises an MCP-ADAR E488QT490A fusion protein. In some embodiments, the alteration in an original composition of the repRNA comprises a sequence edit in the repRNA sequence. In some embodiments, the sequence edit in the repRNA sequence comprises one or more adenosine to inosine conversions in the repRNA sequence. In certain embodiments, the editing array sequence is capable of accumulating sequence edits over time, and wherein determining the number of accumulated sequence edits in the editing array over a time period determines the age of the repRNA. In some embodiments, determining one or more edits in the editing array sequence over a time period indicates a repRNA response to a stimulus. In some embodiments, the number of edits in the editing array sequence corresponds to a temporal record of activation of a promoter that generates the repRNAs, and a number of edits in the repRNA corresponds to the length of time elapsed since the activation of the promoter. In certain embodiments, the number of repRNA edits in a test repRNA compared to the number of repRNA edits in a control repRNA indicates the time since promoter activation of the test repRNA and the time since promoter activation of the control repRNA, wherein a different number of edits in the test repRNA compared to the control repRNA indicates a difference in the time period since the activation by the promoter of the test repRNA compared to the time period since the activation by the promoter of the control repRNA. In some embodiments, the stimulus an electrical stimulus, a chemical stimulus, a biological stimulus, a signaling molecule, a signaling chemical, a temperature stimulus, or a light stimulus. In some embodiments, the stimulus activates a promoter and activation of the promoter generates new repRNAs. In some embodiments, the target RNA encodes a detectable protein. In certain embodiments, the detectable protein comprises a fluorescent protein. In some embodiments, the alteration in the repRNA composition comprises a change in the repRNA sequence, wherein the change comprises one or more of: (i) a modification of one or more nucleic acids in the repRNA RNA sequence, and (ii) one or more of a substitution, deletion, and addition of a nucleic acid in the repRNA RNA sequence. In certain embodiments, the repRNA composition is determined using a sequencing means. In some embodiments, the sequencing means comprises single cell sequencing. In some embodiments, the determined repRNA composition is compared to a control repRNA composition and detection of one or more differences between the repRNA composition and the control repRNA composition indicates a change in the repRNA composition. In certain embodiments, the change in the repRNA comprises one or more adenosine to inosine conversions in the repRNA sequence. In some embodiments, the number of changes in the repRNA composition corresponds to the temporal history of activity of a promoter that generates a population of repRNAs. In some aspects of the invention any embodiment of an aforementioned system is included in a cell. In some embodiments a repRNA is in a cell. In certain embodiments, a predetermined enzyme is also in the cell. In some embodiments, the cell is one or more of: a vertebrate cell, a mammalian cell, and a human cell. In some embodiments, wherein the cell is an excitable cell. In certain embodiments, the cell is one or more of: a neuron, a CNS cell, a PNS cell, a muscle cell, an endocrine cell, an immune system cell, an epidermal cell, a kidney cell, a liver cell, and a cardiac cell. In some embodiments, the cell is an in vitro cell.

According to another aspect of the invention, a cell is provided that includes any embodiment of any aforementioned aspect of an RNA-based molecular recording system. In some embodiments, the cell is one or more of: a vertebrate cell, a mammalian cell, a human cell, an excitable cell, a neuron, a CNS, cell, a PNS cell, a muscle cell, a endocrine cell, an immune system cell, an epidermal cell, a kidney cell, a liver cell, a cardiac cell, and an in vitro cell. In some embodiments, wherein the cell is an in vitro cell.

According to another aspect of the invention, a vector is provided that includes one or both of the repRNA and predetermined enzyme set forth in any embodiment of any aforementioned aspect of an RNA-based molecular recording system.

According to another aspect of the invention, a cell that includes any vector of an aforementioned aspect of the invention. In certain embodiments, the cell is one or more of: a vertebrate cell, a mammalian cell, and a human cell, an excitable cell, a neuron, a CNS, cell, a PNS cell, a muscle cell, an endocrine cell, an immune system cell, an epidermal cell, a kidney cell, a liver cell, a cardiac cell, and an in vitro cell.

According to another aspect of the invention, methods of RNA-based molecular recording in a cell are provided. The methods including one or more of any embodiment of a repRNA and predetermined enzyme of any aspect of an aforementioned RNA-based molecular recorder system, and determining the presence of an alteration in the original composition of the repRNA. In some embodiments, including the repRNA and predetermined enzyme in a cell comprises one of more of: expressing and delivering. In certain embodiments, the editing enzyme is bound to an endogenous RNA. In some embodiments, the editing array is inserted into the 3′-UTR of an endogenous RNA. In some embodiments, the RNA comprises mRNA. In some embodiments, the method also includes determining an age of the RepRNA. In certain embodiments, the method also includes determining a response of the repRNA to an applied stimulus. In some embodiments, a means for the determining comprises detecting one or more alternations in the repRNA composition. In certain embodiments, the alteration in the repRNA composition comprises a change in the repRNA sequence, wherein the change comprises one or more of: (i) a modification of one or more nucleic acids in the repRNA RNA sequence, and (ii) one or more of a substitution, deletion, and addition of a nucleic acid in the repRNA RNA sequence.

Additional aspects of the invention include systems and/or use of RNA as a substrate for molecular recording. In some embodiments systems of the invention are used to determine the age of one or more RNAs by altering the composition of the sequence of the RNA. In certain embodiments, methods of the invention use of a system of the invention to monitor the age of RNA in a cell using RNA editing, which may additionally include using the age of RNAs to infer the transcriptional history of the cell. In certain aspects, the invention includes methods of recording multiple different signals on the same RNA; using stimulus-responsive dimerization domains to record the total amount of a given signal that a cell has observed during the lifetime of a particular RNA; and combining both an age-recording system with a stimulus-responsive dimerization system to make a system that can record the time course the application of arbitrary stimuli into the sequence of the RNA. In certain aspects, the invention includes an RNA-based molecular reporting system in which a predetermined enzyme is attached to an endogenous RNA, and the age of the RNA can then to be determined based on detected modifications to the endogenous nucleotide bases of the RNA sequence. Such an embodiment need not include an engineered editing array and does not require the RNA sequence to be an “engineered” RNA sequence.

Additional aspects of the invention include use of a set of 12 adenosines that were edited with much higher rate constants than other adenosines in the repRNA experiments described herein. These “high-edit” adenosines were found to be edited on almost every RNA observed. Certain embodiments of the invention utilize such high-edit adenosines as result in faster, for example, single-minute time resolutions. In addition, the invention in some aspects includes use of dimerization systems, instead of the constantly-active MCP-MS2 system, to link ADAR or CDAR to constitutively expressed repRNAs in a stimulus-specific manner. In certain aspects of the invention systems are prepared and used to record both age and a stimulus-specific signal on a single repRNA. In addition, the invention includes, in some aspects systems and methods for reporting on timing of other kinds of cellular events such as but not limited to: calcium-related effects and the effect of one or more signaling molecules. The aspects of the invention set forth above, as well as the additional aspects and embodiments set forth elsewhere herein support use of RNA tickertape as a scalable and extensible approach for recording the histories of cells.

### BRIEF DESCRIPTION OF THE DRAWINGS

FIG. 1A-F provides schematic diagrams and graphs of results showing repRNAs are efficiently edited by ADAR E488QT490A. FIG. 1A provides a schematic diagram of the RNA tickertape concept. The temporal history of promoter activity is determined by examination of the distribution of the number of A>I edits per RNA. Prior to promoter activity, the distribution is at steady state (left). A burst of promoter activity generates a population of new, unedited RNAs [distribution of edits per RNA shifts to lower values (center)]. These RNAs are then gradually edited [distribution of edits per RNA shifts to higher values over time (right)]. FIG. 1B is a schematic diagram showing embodiments of reporter RNAs (repRNAs) that include editing arrays of adenosines (grey dots) and several MS2 step loops in the 3′ UTR of an mRNA. In the presence of an MCP-ADAR fusion (MCP is indicated by blobs on the stem loops, while ADAR is indicated by the hexagons), repRNAs are edited over time by catalytic conversion of adenosine to inosine (black dots). FIG. 1C is a schematic showing the structure of a portion of an embodiment of one repRNA, showing MS2 stem loops and the repetitive, double-stranded RNA motif that serves as the editing substrate. An example of a portion of a repRNA sequence is enlarged and boxed with a dashed line (left dashed-line box) to illustrate an initial repRNA sequence (upper sequence, SEQ ID NO: 58; lower sequence, SEQ ID NO: 59). Subsequent A>I editing of the example initial repRNA is illustrated in a second box (right dashed-line box) (upper sequence, SEQ ID NO: 60; lower sequence, SEQ ID NO: 61; In=inosine). The region shown is in the 3′ UTR of an iRFP transcript. FIG. 1D is a schematic representation of an embodiment of a Tet-responsive tickertape system and experimental timeline. FIG. 1E provides histograms illustrating that transcription by the TRE promoter was induced by doxycycline, and was stopped by actinomycin D one hour later. As in the schematic of FIG. 1D, doxycycline induction shifted the editing distribution towards lower values as new RNAs were generated. After promoter activity ceased, the repRNAs accumulated edits and the distribution moved to higher values. All histograms are normalized so the sum of all values is 1. FIG. 1F is a graph showing mean edits per transcript for TRE induction as a function of time for an embodiment of the TRE tickertape system. Error bars show standard deviation (s.d.), N=3 technical replicates.

FIG. 2A-C provides a schematic diagram of embodiments of templates, graphs, and histograms. FIG. 2A provides a diagram showing five editing templates (Templates A-E) that were tested. Template A comprises SEQ ID NOs: 62-63, upper and lower, respectively; Template B comprises SEQ ID NOs: 64-65, upper and lower, respectively; Template C comprises SEQ ID NOs: 66-67, upper and lower, respectively; Template D comprises SEQ ID NOs: 68-69, upper and lower, respectively; and Template E comprises SEQ ID NOs: 70-71, upper and lower, respectively. Of these five templates, templates A and B showed robust temporal editing that seemed appropriate for the construction of the tickertape system. Notes on the templates are provided in Table 3. FIG. 2B provides graphs showing the mean number of edits per RNA for several different time points for three different ADAR variants, and for templates A and B. The protocol used in this study was identical to that used in study illustrated in FIG. 1E. Some combinations, such as dmE488Q with template A, may show greater temporal resolution at short timescales. FIG. 2C provides example editing histograms for three different time points, for each combination of the three enzymes and two templates in FIG. 2B.

FIG. 3A-B provides histograms and a graph of results of studies in which (FIG. 3A) cells were induced with doxycycline, followed by actinomycin D 1 hour later, and then lysis 7 hours after actinomycin D. All editing histograms are normalized to sum to 1. FIG. 3A, top histogram row on left: the editing histogram for cells that were not transfected with ADAR, without removing RNAs with no edits on read 1 or read 2 (i.e., “with zeros”). FIG. 3A, top histogram row on right: the editing histogram for cells that were transfected with ADAR, without removing RNAs with no edits on read 1 or read 2. FIG. 3A bottom histogram row is same as top row, but only considering RNAs with at least one edit on both Read 1 and Read 2 (i.e., “without zeros,” see Methods in Examples section). FIG. 3B is a graph in which qPCR for the iRFP transcript, normalized to GAPDH, is shown as a function of time during the experiment shown in FIG. 1E. Values are normalized to the pre-doxycycline time point. Error bars show standard deviation (N=3).

FIG. 4A-D provides schematic diagrams and histograms of an embodiment of repRNA. FIG. 4A is a diagram of the read structure of the repRNA. FIG. 4B is a schematic of the analysis pipeline. See Methods in Example section. FIG. 4C is for one replicate from the experiment in FIG. 1E, and provides a histogram of the number of reads with a given percentage of As with Q score>27. This includes all sites that are As on the repRNA template, i.e., it also counts Gs that are read at positions that are A on the template. The black vertical line indicates the 90% cutoff, which was applied to all analysis. FIG. 4D is a histogram for one replicate from the experiment in FIG. 1E. The percentage of reads having no edits in either R1 or R2 is shown as a function of time. These reads were excluded from analysis, except where otherwise stated in FIG. 3.

FIG. 5A-I provides graphs and histograms illustrating inference of the timing of promoter activity using an embodiment of RNA tickertape. All editing histograms are normalized to sum to 1. FIG. 5A is a graph in which the fraction of A>I edits as a function of time is shown for three different bases on the repRNA, data from one replicate of FIG. 1E. Best exponential fits are shown. The black dotted line indicates the addition of actinomycin D. FIG. 5B is a plot showing results for the same replicate as in FIG. 5A. The R² value of the exponential fit is shown for each base on the transcript. The black dotted line indicates the R²>0.9 cutoff used for the exponential model. FIG. 5C shows the masked editing histograms for four time points from the same replicate (only the bases with R²>0.9 are included). In each the curved line shows the Poisson binomial distribution for each time point including all the bases with R²>0.9 (see Methods in Examples section). FIG. 5D is a histogram showing the masked (R²>0.9 in all 3 replicates from FIG. 1E, see Methods in Examples section) editing histogram for a single 2.5 hour replicate along with Poisson binomial distribution for 2.5 hours (black line), and the Poisson binomial distribution with least KL divergence from the empirical distribution (gray line). The time estimate is mean±s.d. (N=3 technical replicates). FIG. 5E, as in FIG. 5D shows an editing histogram, but for the 4.5 hour time point. FIG. 5F is a histogram in which the mean absolute error is shown for FIG. 5D (on left) and FIG. 5E (on right). Error bars show standard deviation. FIG. 5G provides at top the editing histograms for 100,000 cells from two time points (nominally 4 and 8 hours, see Methods), group 1 and group 2. FIG. 5G at bottom shows the editing histograms for three representative single cells from group 1 and group 2. FIG. 5H provides time point estimates for each single cell (dots) from group 1 and group 2. Within each box, the upper line shows the mean single cell estimate (reported value is mean±s.d.), and the lower line shows the bulk estimate. Outer error bars show range, while box shows 25^{th} and 75^{th} percentile. Group 1, N=9 cells; group 2, N=10 cells. FIG. 5I shows the mean absolute error in the estimates for the single cells. Except in FIG. 5H, all error bars show s.d.

FIG. 6A-F provides graphs and histograms of results indicating that tickertape can decode arbitrary programs of transcriptional activity. All editing histograms are normalized to sum to 1. FIG. 6A provides results indicating that arbitrary temporal patterns of transcriptional activity (top left) can be recorded into histograms of the number of edits per repRNA (bottom left). In order to recover this information, arbitrary histograms can be modeled as convex sums of the one-hour distributions observed in the TRE tickertape experiments (middle). An approximation of the true history of transcriptional activity is recovered using gradient descent (top right) to minimize the difference between the observed editing distribution and the convex sum (bottom right). FIG. 6B (left) provides simulated editing histogram generated from two 2-hour bursts of transcriptional activity. The approximation recovered by the gradient descent algorithm is shown in FIG. 6B (right). FIG. 6C provides a graph showing the median error (given as |A-G|/G, where A is the approximated weight and G is the ground truth weight) in the weights found by the gradient descent over 1000 trials is shown as a function of time, for different time resolutions (see Methods in Examples section). In the case of 2hr and 3hr resolutions, the 2 hr and 3 hr running averages were calculated and then analyzed identically to the 1 hr case. FIG. 6D provides a graph showing the mean of the errors in FIG. 6C over time points for different time resolutions. FIG. 6E shows reconstruction of transcriptional programs by the gradient descent algorithm for cells that were stimulated with doxycycline for three hours prior to lysis. The empirical editing distribution (FIG. 6E top) is observed from the presumed theoretical weights (FIG. 6E bottom left) and the associated to weights found by the gradient descent are in FIG. 6E, bottom right (N=3 technical replicates). FIG. 6F, shows results as in FIG. 6E, but the cells were stimulated for six hours prior to lysis. All error bars show s.d.

FIG. 7A-C shows results from an embodiment of the Vivid Tickertape system. FIG. 7A shows editing histograms for 3T3 cells transfected with repRNAs expressed under the Vivid promoter, and stimulated for 1 hour (see Methods in Examples section). The editing histogram for cells lysed one hour after stimulation began (i.e., immediately after it ended) is shown in dark gray, and the histogram for cells lysed 6 hours after stimulation began is shown in light gray. All editing histograms are normalized to sum to 1. FIG. 7B is a graph showing the mean number of edits per RNA for the time points generated. Time indicates number of hours since the beginning of stimulation (the first time point is pre-stimulation). Error bars are standard deviation (N=3). FIG. 7C is a graph showing the absolute prediction error, in minutes, averaged over all replicates for the 2 hr, 3 hr, and 4 hr time points. The prediction was performed by mean interpolation, analogously to FIG. 8D. Error bar is standard deviation (N=9, 3 replicates at each of 3 time points).

FIG. 8A-E provides a schematic diagrams, histograms and graphs of results obtained in an embodiment of sequencing-based activity measurement in neurons using c-Fos tickertape methods of the invention. FIG. 8A is a schematic of tickertape constructs and experimental timeline for neuronal recording. FIG. 8B provides editing histograms for neurons prior (dark gray) and one hour following (light gray) KCl induction. The lower overall editing rate for the +KCl case indicates the generation of new repRNAs by the c-fos promoter. Editing histograms are normalized so the sum of all values is 1. FIG. 8C is a graph showing the mean editing rate as a function of time following KCl induction. FIG. 8D is a graph showing the predicted and actual time estimates for all time points. Dotted line is a guide for Y=X. There are no estimates for the 1 hour and 7 hour time points due to mean interpolation. In FIG. 8E the mean absolute error in the predictions from FIG. 8C is shown as a function of time since induction. All error bars show s.d. (N varies, see Methods in Examples section).

FIG. 9A-B provides a chart and histograms illustrating results of an embodiment of multiplexing with the Tickertape system. FIG. 9A is a chart showing results from cells transfected with a barcoded TRE-responsive repRNA construct, a barcoded Vivid-responsive repRNA construct, or both. The number of reads for the TRE-responsive repRNA, Vivid-responsive repRNA, or both are shown. When only one repRNA is transfected, only one barcode is detected in significant numbers, confirming that there is minimal crossover between repRNA barcodes. Note that the third column is not the sum of the first and second columns, because it includes barcodes that did not perfectly align to either the Tet or Vivid repRNA barcodes. To further confirm the possibility of multiplexing using barcoded repRNAs, editing histograms were analyzed for cells that were transfected with a barcoded TRE-responsive repRNA construct, a barcoded Vivid-responsive repRNA construct, or both (FIG. 9B). The editing histograms for the Vivid-responsive and TRE-responsive repRNAs do not seem to change when the other repRNA is also present, indicating there is minimal cross-talk between barcoded repRNA constructs. All editing histograms are normalized to sum to 1.

FIG. 10A-F provides histograms showing performance of the gradient descent algorithm. For 1000 randomly generated weight vectors (“simulated vectors”), gradient descent was used to find the approximation (“approximated vectors”) that minimized the L2 norm (“inner product”) between the RNA editing distribution corresponding to the simulated vectors (“simulated distributions”) and the RNA editing distribution corresponding to the approximated vectors (“approximated distributions”). The L2 norm between the distributions is referred to as the inner product to distinguish it from the L2 norm between the vectors, which is referred to as the mean squared error (MSE). In FIG. 10A the inner product between simulated distributions and approximated distributions is shown in the left histogram (dark gray). By contrast, the inner product between simulated distributions and other random distributions is shown in the right histogram (light gray). In FIG. 10B the mean squared error between the simulated vectors and approximated vectors is shown in dark gray. By contrast, the inner product between the simulated distributions and other random distributions is shown in light gray. Note that a substantial number of random weight vectors have lower mean squared error than the approximated vectors. This is possible because the noise in the basis distribution set used to generate the approximated distributions from the approximated vectors is different from the noise in the basis distribution set used to generate the simulated distributions from the simulated vectors, so the minimum of inner product between the simulated and approximated distributions is not always the same as the minimum of the MSE between the simulated and approximated vectors. FIG. 10C provides another visualization of FIG. 10B. For each simulated vector, both an approximated vector and a random vector were calculated. The difference in MSE between the approximated and random vectors is shown. Negative values correspond to test vectors for which the associated random vector was a better approximation to the simulated vector than the approximated vector. In FIG. 10D, dark gray and light gray bars are the same as in FIG. 10B. White bars correspond to the minimum MSE among all of the solutions found by gradient descent for a given test vector, indicating that the inner product minima found by the gradient descent are not in general minima of the MSE. FIG. 10E shows the difference in the inner product between the solutions with the minimum MSE found by gradient descent, and the solutions with the minimum inner product, as a fraction of the minimum inner product. The solutions with the minimum MSE discovered by gradient descent often have inner products several-fold higher than the solution with the minimum inner product. FIG. 10F is equivalent to FIG. 6D, but comparing test distributions to other random test distributions, rather than to minima of the inner product obtained by gradient descent. Error bars show s.d.

### BRIEF DESCRIPTION OF CERTAIN OF THE SEQUENCES

Table 1 provides a list of oligonucleotides used in embodiments of RNA-based molecular recording systems set forth herein.

TABLE 1
Oligonucleotides (SEQ ID NOs: 1-50)

| | | | SEQ |
|---|---|---|---|
| Name | Description | Sequence | ID NO |
| SGR-174B-1 | Barcoded | aatgatacggcgaccaccgagatctacacnnnnnnnnnnnncctgcgagg | 1 |
| | RT Primer | cccgcatctttcacaaattttgtaatccagagg | |
| | with 3bp | | |
| | barcode | | |
| SGR-174B-2 | Barcoded | aatgatacggcgaccaccgagatctacacnnnnnnnnnnnngaggcgagg | |
| | RT Primer | cccgcatctttcacaaattttgtaatccagagg | 2 |
| | with 3bp | | |
| | barcode | | |
| SGR-174B-3 | Barcoded | aatgatacggcgaccaccgagatctacacnnnnnnnnnnnnttagcgagg | 3 |
| | RT Primer | cccgcatctttcacaaattttgtaatccagagg | |
| | with 3bp | | |
| | barcode | | |
| SGR-174B-4 | Barcoded | aatgatacggcgaccaccgagatctacacnnnnnnnnnnnnagcgcgagg | 4 |
| | RT Primer | cccgcatctttcacaaattttgtaatccagagg | |
| | with 3bp | | |
| | barcode | | |
| SGR-174B-5 | Barcoded | aatgatacggcgaccaccgagatctacacnnnnnnnnnnnnaatgcgagg | 5 |
| | RT Primer | cccgcatctttcacaaattttgtaatccagagg | |
| | with 3bp | | |
| | barcode | | |
| SGR-174B-6 | Barcoded | aatgatacggcgaccaccgagatctacacnnnnnnnnnnnncaagcgagg | 6 |
| | RT Primer | cccgcatctttcacaaattttgtaatccagagg | |
| | with 3bp | | |
| | barcode | | |
| SGR-174B-7 | Barcoded | aatgatacggcgaccaccgagatctacacnnnnnnnnnagtgtcgcgagg | 7 |
| | RT primer | cccgcatctttcacaaattttgtaatccagagg | |
| | with 6 base | | |
| | barcode | | |
| SGR-174B-8 | Barcoded | aatgatacggcgaccaccgagatctacacnnnnnnnnntatccggcgagg | 8 |
| | RT primer | cccgcatctttcacaaattttgtaatccagagg | |
| | with 6 base | | |
| | barcode | | |
| SGR-174B-9 | Barcoded | aatgatacggcgaccaccgagatctacacnnnnnnnnncatttggcgagg | 9 |
| | RT primer | cccgcatctttcacaaattttgtaatccagagg | |
| | with 6 base | | |
| | barcode | | |
| SGR-174B-10 | Barcoded | aatgatacggcgaccaccgagatctacacnnnnnnnnnatgctagcgagg | 10 |
| | RT primer | cccgcatctttcacaaattttgtaatccagagg | |
| | with 6 base | | |
| | barcode | | |
| SGR-174B-11 | Barcoded | aatgatacggcgaccaccgagatctacacnnnnnnnnnccgtgggcgagg | 11 |
| | RT primer | cccgcatctttcacaaattttgtaatccagagg | |
| | with 6 base | | |
| | barcode | | |
| SGR-174B-12 | Barcoded | aatgatacggcgaccaccgagatctacacnnnnnnnnnatgagtgcgagg | 12 |
| | RT primer | cccgcatctttcacaaattttgtaatccagagg | |
| | with 6 base | | |
| | barcode | | |
| SGR-174B-13 | Barcoded | aatgatacggcgaccaccgagatctacacnnnnnnnnncgagcagcgagg | 13 |
| | RT primer | cccgcatctttcacaaattttgtaatccagagg | |
| | with 6 base | | |
| | barcode | | |
| SGR-174B-14 | Barcoded | aatgatacggcgaccaccgagatctacacnnnnnnnnncgcggcgcgagg | 14 |
| | RT primer | cccgcatctttcacaaattttgtaatccagagg | |
| | with 6 base | | |
| | barcode | | |
| SGR-174B-15 | Barcoded | aatgatacggcgaccaccgagatctacacnnnnnnnnnacttatgcgagg | 15 |
| | RT primer | cccgcatctttcacaaattttgtaatccagagg | |
| | with 6 base | | |
| | barcode | | |
| SGR-174B-16 | Barcoded | aatgatacggcgaccaccgagatctacacnnnnnnnnntgcatggcgagg | 16 |
| | RT primer | cccgcatctttcacaaattttgtaatccagagg | |
| | with 6 base | | |
| | barcode | | |
| SGR-174B-17 | Barcoded | aatgatacggcgaccaccgagatctacacnnnnnnnnnagtagggcgagg | 17 |
| | RT primer | cccgcatctttcacaaattttgtaatccagagg | |
| | with 6 base | | |
| | barcode | | |
| SGR-174B-18 | Barcoded | aatgatacggcgaccaccgagatctacacnnnnnnnnngttgacgcgagg | 18 |
| | RT primer | cccgcatctttcacaaattttgtaatccagagg | |
| | with 6 base | | |
| | barcode | | |
| SGR-174B-19 | Barcoded | aatgatacggcgaccaccgagatctacacnnnnnnnnntatcacgcgagg | 19 |
| | RT primer | cccgcatctttcacaaattttgtaatccagagg | |
| | with 6 base | | |
| | barcode | | |
| SGR-174B-20 | Barcoded | aatgatacggcgaccaccgagatctacacnnnnnnnnnccctaggcgagg | 20 |
| | RT primer | cccgcatctttcacaaattttgtaatccagagg | |
| | with 6 base | | |
| | barcode | | |
| SGR-174B-21 | Barcoded | aatgatacggcgaccaccgagatctacacnnnnnnnnngcccgtgcgagg | 21 |
| | RT primer | cccgcatctttcacaaattttgtaatccagagg | |
| | with 6 base | | |
| | barcode | | |
| SGR-174B-22 | Barcoded | aatgatacggcgaccaccgagatctacacnnnnnnnnnttcccggcgagg | 22 |
| | RT primer | cccgcatctttcacaaattttgtaatccagagg | |
| | with 6 base | | |
| | barcode | | |
| SGR-174B-23 | Barcoded | aatgatacggcgaccaccgagatctacacnnnnnnnnncatatagcgagg | 23 |
| | RT primer | cccgcatctttcacaaagtaatccagagg | |
| | with 6 base | | |
| | barcode | | |
| SGR-174B-24 | Barcoded | aatgatacggcgaccaccgagatctacacnnnnnnnnnaacgccgcgagg | 24 |
| | RT primer | cccgcatctttcacaaattagtaatccagagg | |
| | with 6 base | | |
| | barcode | | |
| SGR-174B-25 | Barcoded | aatgatacggcgaccaccgagatctacacnnnnnnnnnaggttggcgagg | 25 |
| | RT primer | cccgcatctttcacaaattagtaatccagagg | |
| | with 6 base | | |
| | barcode | | |
| SGR-174B-26 | Barcoded | aatgatacggcgaccaccgagatctacacnnnnnnnnntcaatagcgagg | 26 |
| | RT primer | cccgcatctttcacaaattagtaatccagagg | |
| | with 6 base | | |
| | barcode | | |
| SGR-175 | Custom | gcgaggcccgcatctttcacaaattttgtaatccagagg | 27 |
| | Read 1 | | |
| SGR-175-RC | Custom | cctctggattacaaaatttgtgaaagatgcgggcctcgc | 28 |
| | Index 2 | | |
| SGR-176 | Barcoded | caagcagaagacggcatacgagatactggtcaaagttactatcgaaatgc | 29 |
| | PCR primer | cctgagtccaccccgg | |
| SGR-176-2 | Barcoded | caagcagaagacggcatacgagatgtgttcgtaagttactatcgaaatgc | 30 |
| | PCR primer | cctgagtccaccccgg | |
| SGR-176-3 | Barcoded | caagcagaagacggcatacgagattaactgttaagttactatcgaaatgc | 31 |
| | PCR primer | cctgagtccaccccgg | |
| SGR-176-4 | Barcoded | caagcagaagacggcatacgagatgattggtgaagttactatcgaaatgc | 32 |
| | PCR primer | cctgagtccaccccgg | |
| SGR-176-5 | Barcoded | caagcagaagacggcatacgagatggagagagaagttactatcgaaatgc | 33 |
| | PCR primer | cctgagtccaccccgg | |
| SGR-176-6 | Barcoded | caagcagaagacggcatacgagattgagcgataagttactatcgaaatgc | 34 |
| | PCR primer | cctgagtccaccccgg | |
| SGR-176-7 | Barcoded | caagcagaagacggcatacgagatcctccgttaagttactatcgaaatgc | 35 |
| | PCR primer | cctgagtccaccccgg | |
| SGR-176-8 | Barcoded | caagcagaagacggcatacgagataacatattaagttactatcgaaatgc | 36 |
| | PCR primer | cctgagtccaccccgg | |
| SGR-176-9 | Barcoded | caagcagaagacggcatacgagatcttacgtaaagttactatcgaaatgc | 37 |
| | PCR primer | cctgagtccaccccgg | |
| SGR-176-10 | Barcoded | caagcagaagacggcatacgagattgacgtagaagttactatcgaaatgc | 38 |
| | PCR primer | cctgagtccaccccgg | |
| SGR-176-11 | Barcoded | caagcagaagacggcatacgagatctatgtataagttactatcgaaatgc | 39 |
| | PCR primer | cctgagtccaccccgg | |
| SGR-176-12 | Barcoded | caagcagaagacggcatacgagattttgcagaaagttactatcgaaatgc | 40 |
| | PCR primer | cctgagtccaccccgg | |
| SGR-176-13 | Barcoded | caagcagaagacggcatacgagatggtagcgaaagttactatcgaaatgc | 41 |
| | PCR primer | cctgagtccaccccgg | |
| SGR-176-14 | Barcoded | caagcagaagacggcatacgagatacgggtttaagttactatcgaaatgc | 42 |
| | PCR primer | cctgagtccaccccgg | |
| SGR-176-15 | Barcoded | caagcagaagacggcatacgagattaaacctcaagttactatcgaaatgc | 43 |
| | PCR primer | cctgagtccaccccgg | |
| SGR-176-16 | Barcoded | caagcagaagacggcatacgagatgagaactgaagttactatcgaaatgc | 44 |
| | PCR primer | cctgagtccaccccgg | |
| SGR-176-17 | Barcoded | caagcagaagacggcatacgagatggtttgataagttactatcgaaatgc | 45 |
| | PCR primer | cctgagtccaccccgg | |
| SGR-176-18 | Barcoded | caagcagaagacggcatacgagattagattataagttactatcgaaatgc | 46 |
| | PCR primer | cctgagtccaccccgg | |
| SGR-176-19 | Barcoded | caagcagaagacggcatacgagataaggttagaagttactatcgaaatgc | 47 |
| | PCR primer | cctgagtccaccccgg | |
| SGR-176-20 | Barcoded | caagcagaagacggcatacgagatccgaaaataagttactatcgaaatgc | 48 |
| | PCR primer | cctgagtccaccccgg | |
| SGR-177 | Custom | aagttactatcgaaatgccctgagtccaccccgg | 49 |
| | Read 2 | | |
| SGR-177-RC | Custom | ccggggtggactcagggcatttcgatagtaactt | 50 |
| | Index 1 | | |

Table 2 provides a list of RNA editing templates used in embodiments of RNA-based molecular recording systems set forth herein.

TABLE 2
Oligonucleotides (SEQ ID NOs: 51-57)

| Name | | |
|---|---|---|
| and SEQ | | |
| ID NO | Sequence | Notes |
| A_Short | agtacgcgttagattagattagattagattagattagattagaa | |
| SEQ ID | aaattaatacgtacaccatcagggtacgtctcagacaccatcag | |
| NO: 51 | ggtctgtctggtacagcatcagcgtaccatatattttttccaat | |
| | ccaatccaatccaatccaatccaatccaaatagatcctaatca | |
| A | ttagattagattagattagattagattagattagaaaaattaat | |
| SEQ ID | atacgtacaccatcagggtacgtcatatattttttccaatccaa | |
| NO: 52 | tccaatccaatccaatccaatccaatacgcgttagattagatta | |
| | gattagattagattagattagaaaaattaatacgtacaccatca | |
| | gggtacgtctcagacaccatcagggtctgtctggtacagcatca | |
| | gcgtaccatatattttttccaatccaatccaatccaatccaatc | |
| | caatccaaatagatcctaatca | |
| B_Short | agtacgcgttagattagattagattagattagattagattagaa | |
| SEQ ID | aaattaatacgtacaccatcagggtacgtctcagacaccatcag | |
| NO: 53 | ggtctgtctggtacagcatcagcgtaccatatattttttctaat | |
| | ctaatctaatctaatctaatctaatctaaatagatcctaatca | |
| B | ttagattagattagattagattagattagattagaaaaattaat | |
| SEQ ID | atacgtacaccatcagggtacgtcatatattttttctaatctaa | |
| NO: 54 | tctaatctaatctaatctaatctaaacgcgttagattagattag | |
| | attagattagattagattagaaaaattaatacgtacaccatcag | |
| | ggtacgtctcagacaccatcagggtctgtctggtacagcatcag | |
| | cgtaccatatattttttctaatctaatctaatctaatctaatct | |
| | aatctaaatagatcctaatca | |
| C | agtacgcgttaaattatattaactaaattatagattaacaagaa | This template shows significant background |
| SEQ ID | tattaaatacgtacaccatcagggtacgtctcagacaccatcag | editing by endogenous ADAR enzymes, even in |
| NO: 55 | ggtctgtctggtacagcatcagcgtacctatttaatattcttgt | the absence of transexpression of ADAR. It |
| | taatctataatttagttaatataatttaaatagatcctaatca | also showed extremely rapid editing on a |
| | | timescale of single minutes in the presence |
| | | of blue light, when MCP-Cry2 and CIBN- |
| | | dmADARE488Q were co-expressed. |
| D | agtacgcgattggttaatcccattggttaatcccattggttaat | Editing on this template showed significant |
| SEQ ID | cccttaatacgtacaccatcagggtacgtctcagacaccatcag | sensitivity to the identity of the N- |
| NO: 56 | ggtctgtctggtacagcatcagcgtaccatatatgggttaaact | terminal fusion. MCP-ADAR was able to edit |
| | gatgggttaaactgatgggttaaactgatatagatcctaatca | this template, whereas other ADAR enzymes, |
| | | like a C1BN-ADAR fusion, were unable. |
| E | agtagcgaaaaaaaaaaaaaaaaaaaaaaaaaaaaaaaaaaaaa | This template was always severely under- |
| SEQ ID | aaaaaaaacgtacaccatcagggtacgtctcagacaccatcagg | represented in sequencing, either due to |
| NO: 57 | gtctgtctggtacagcatcagcgtaccttttttttttttttttt | difficulties with expression, amplification, |
| | ttttttttttttttttttttttttttttatagatcctaatca | or alignment. |

### DETAILED DESCRIPTION

A new class of molecular recorders based on RNA has now been designed and developed. An RNA-based molecular recorder of the invention, also referred to herein as a “tickertape system” of the invention may be thought of as a temporal microscope, which, rather than mapping point sources onto Airy functions via an objective lens, maps instantaneous transcriptional events onto Poisson binomial editing distributions via the statistical dispersion intrinsic to Poisson processes. Analogous to how fluorescent reporter proteins allow for non-invasive spatial readout of cell state by imaging, reporter RNAs of the invention allow for non-invasive temporal readout of cell state by sequencing. However, whereas the spatial transfer function of a microscope with finite numerical aperture is not invertible, the temporal transfer function between transcription space and editing space is in principle invertible, so the ability of tickertape methods and systems of the invention to infer arbitrary temporal functions of transcriptional activity is not limited by any fundamental diffraction-like limit. Instead, RNA tickertape systems of the invention are limited only by how well the observed editing distribution approximates the true editing distribution, which is a statistical sampling problem. By increasing the number of observations per cell (for example by increasing the number of editing sites per RNA), the accuracy achieved both in bulk and in the single cell case may be made substantially higher.

Embodiments of systems and methods of the invention can be used in cell culture, in vitro preparations, and in in vivo settings. Some aspects of the invention include use of RNA-based molecular reporter systems and components for determining in one or more cells one or more of events such as but not limited to: (i) the timing of transcriptional activity in the cell, and (ii) the presence or absence of an effect resulting from a stimulus received by the cell on transcription in the cell. Embodiments of methods of the invention can be used to record the timings of transcriptional events in real time coordinates in single cells, a plurality of cells, tissues, and organisms.

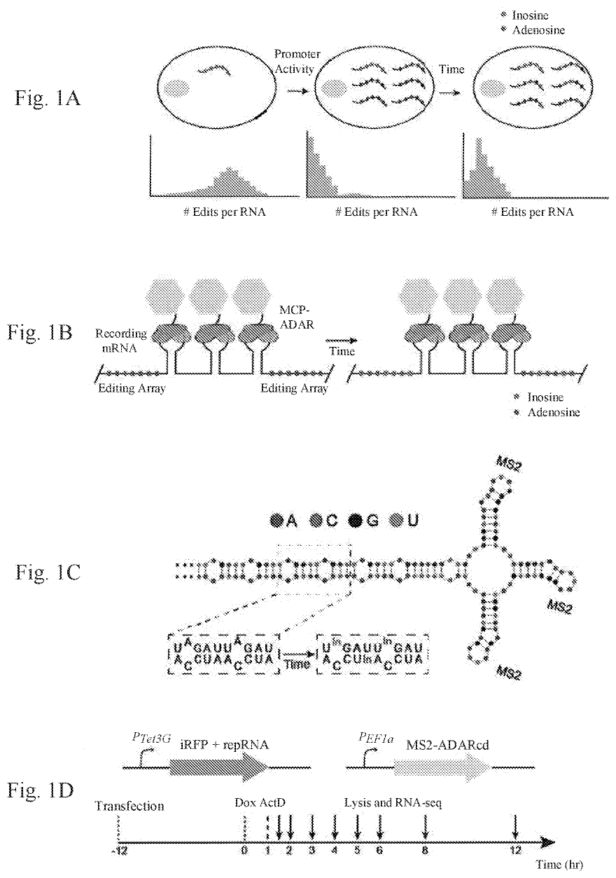

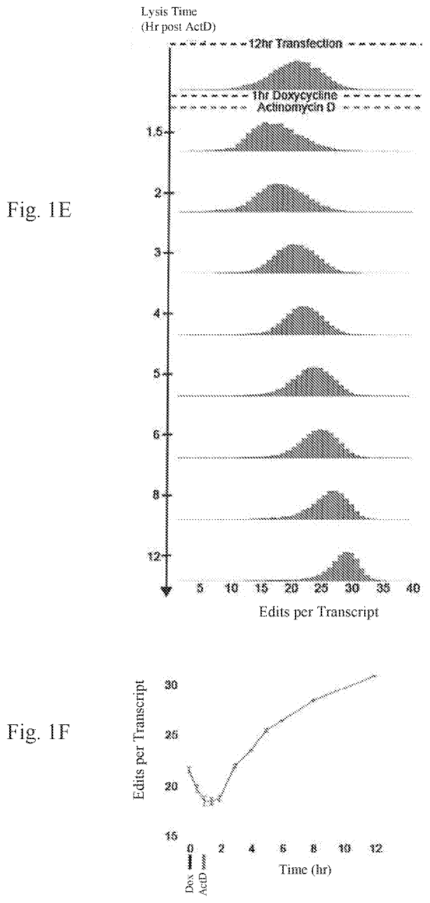

Embodiments of molecular recorder systems of the invention are capable of recording the timings of transcriptional events in real time coordinates. It has now been determined that the history of the activity of a given promoter can be inferred from the distribution of ages of the RNAs generated by that promoter (FIG. 1A). This approach has two key advantages: first, transcription is the direct downstream consequence of many cellular events, so the time resolution of an RNA-based recorder is limited only by the speed at which RNAs can be generated, which is on the order of minutes (11). Second, this approach allows for the straightforward extraction of both the timing and the magnitude of transcriptional events, providing a temporal “activity trace” of the promoter. Conceptually, if reporters of the invention accumulate 1 edit per hour, then a population of 50 RNAs with 10 edits each corresponds to an event 10 hours ago, and a population of 10 RNAs with 5 edits each corresponds to an event 5 hours ago, with one fifth the magnitude.

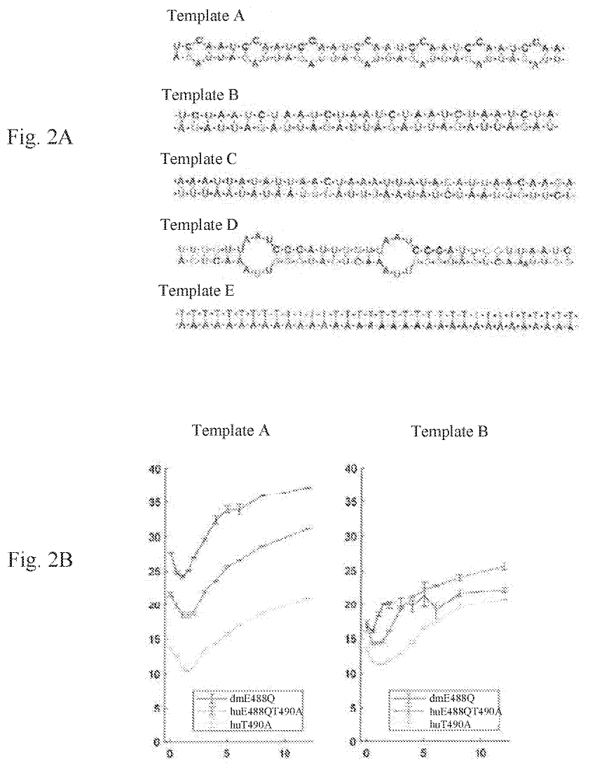

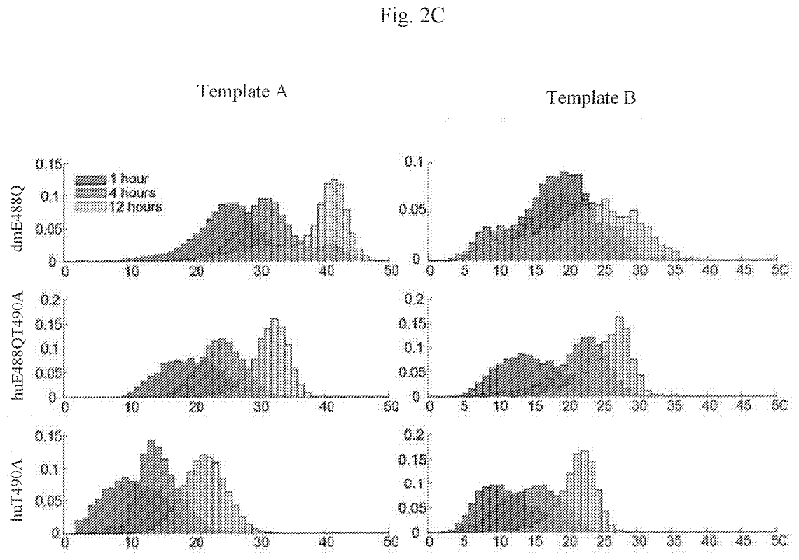

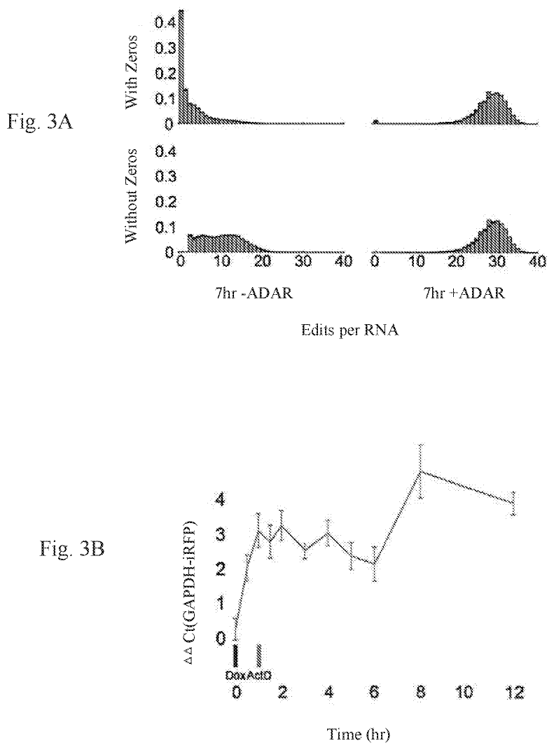

In one embodiment of a system of the invention, reporter RNAs (repRNAs) have been designed and produced that are capable of reporting their age via the gradual accumulation of edits, for example though not intended to be limiting, A→I edits caused by a modified version of the human Adenosine Deaminase Acting on RNA 2 (ADAR2) enzyme (FIG. 1B). In some embodiments, repRNAs of the invention include adenosine-rich editing arrays, in the 3′ UTR of a mRNA (12), that are designed to be favored substrates of the ADAR enzyme (13-15) (FIG. 1C). Edits in this region can subsequently be identified as A→G mutations in RNA sequencing via comparison with the known starting sequence. In certain aspects of the invention an ADAR predetermined enzyme is fused to an MS2 Capsid Protein (MCP) which binds to MS2 binding sites that have been engineered into the editing region of a target mRNA to increase editing activity by local concentration enhancement (16). Multiple repRNA and ADAR variants have now been screened. Certain embodiments of the invention include a pair of repRNA and ADAR variants for which the editing in cells, a non-limiting example of which is a HEK239T cell, occurs over hours, a timescale relevant for most endogenous transcriptional activity (FIG. 2). It has now been confirmed that the bulk of the editing thus observed is due to the MCP-ADAR fusion, rather than endogenous ADAR (FIG. 3A), and that repRNAs do not degrade over the 12 hour observation time (FIG. 3B), so information encoded into the repRNAs is not lost due to RNA degradation. In some aspects of the invention an RNA-based molecular recorder system (also referred to herein as an RNA tickertape system) comprises a repRNA system component and a predetermined enzyme system component. In some embodiments of the invention, a predetermined enzyme may be an ADAR or a CDAR protein, which may be part of a fusion protein that also includes a binding protein, for example, but not limited to: an MCP protein. A non-limiting example of a fusion protein that may be used in some embodiments of the invention is an MCP-ADAR fusion protein, an MCP-CDAR fusion protein, and an MCP-ADAR E488QT490A fusion protein. As described in more detail elsewhere herein, the MCP-ADAR E488QT490A protein is a variant that has catalytic activity comparable to wild-type ADAR but that has reduced adenosine base-flipping activity (14).

Previous systems for temporally resolved detection of neural activity in single cells have relied on methods such as optical detection, or the detection of electric or magnetic fields, and, therefore it has been challenging or not possible to record from many neurons simultaneously, or from deep neural populations. Although the time resolution of RNA tickertape is intrinsically limited by the speed of RNA transcription, it has now been demonstrated that tickertape can be used to perform a sequencing-based readout of the history of activity in a population of neurons with temporal resolution comparable to that of immediate early genes, which are popular for detection of neurons recently active in a neural network, but are primarily used to perform such measurements at single time points (19).

The experimental analysis described herein has in part excluded a set of 12 adenosines that were identified as being edited with much higher rate constants than the other adenosines on the tested repRNAs. The 12 adenosines were identified as being edited on almost every RNA observed. Additional aspects of the invention can take advantage of these rapidly edited RNAs to achieve faster resolution, for example, though not intended to be limiting, single-minute time resolutions. In other aspects of the invention an alternative dimerization system may be used, instead of the constantly-active MCP-MS2 system, in order to link an ADAR or CDAR to constitutively expressed repRNAs in a stimulus-specific manner, or to record both age and a stimulus-specific signal on a single repRNA. In some aspects of the invention tickertapes are constructed that can be used to report on the timing of other kinds of cellular events, non-limiting examples of which are: the impact of contact with calcium or signaling molecules. Embodiments of RNA tickertape systems (also referred to herein as RNA-based molecular recorder systems) can be used as a scalable and extendible approach to record and determine the histories of cells.

Methods to prepare and use system components of the invention such as a repRNA, a predetermined enzyme, and fusion proteins are described herein and also may include art-known methods to deliver and express encoded molecules.

Certain aspects of the invention comprise methods for inclusion and use of RNA-based molecular recorders of the invention in one or more cells, which permits detection in one or more cells. Embodiments of RNA-based molecular recorder systems of the invention can be introduced in specific cells (e.g., using a virus, vector, or other means for delivery) and used to assess stimuli that impact RNA transcription in the cells. The cells may be in intact organisms (including humans) as well as cells in vitro, and in cells in culture.

### Molecules, Expression, and Functions

Certain embodiments of RNA-based molecular recorders of the invention include a repRNA and a predetermined enzyme in a cell. Using such a system of the invention permits determination of one or more alterations that take place in the composition of the repRNA. As used herein, the term: “composition of the repRNA” means the sequence components of the repRNA. An alternation in one or more sequence components of a repRNA means an alteration in the nucleic acid sequence or in a feature of one or more of the nucleic acids in the sequence. For example, a change in a nucleic acid sequence may be a nucleotide insertion, deletion, or substitution, (such as but not limited to an A→G substitution). A non-limiting example of an alteration in a feature of a nucleic acid is a modification of the base itself, rather than a change in the sequence per se. It will be understood that an alteration in the composition of a repRNA may include one or more sequence changes and/or a modification of one or more bases in the repRNA sequence. Various types of base modifications are well known the art.

Embodiments of the invention include molecular recording systems as well as components thereof. For example, components of a molecular recording or tickertape system of the invention include but are not limited to, repRNAs and predetermined enzyme compositions. In some embodiments, a repRNA of the invention includes an editing array in the 3′ UTR of a target RNA. As used herein the term “editing array” refers to a sequence array that includes at least one of (i) one or more engineered binding sites; and (ii) one or more selectively favored substrates of the predetermined enzyme. In certain embodiments of methods of the invention, a predetermined enzyme is selected along with its favored substrate to which it specifically binds and the specifically favored substrate is included in an editing array that is part a repRNA. A target RNA may in some aspects of the invention be an exogenous RNA that is designed and delivered into a cell and in certain aspects of the invention a target RNA is an endogenous RNA that naturally occurs in a cell. A target RNA may be an mRNA, a modified RNA, an RNA with one or more sequence or base variations with respect to RNAs described herein.

Non-limiting examples of editing arrays are: adenosine-rich editing array and a cytosine-rich editing array. In some aspects of the invention, a repRNA includes an engineered binding site positioned in an editing array of the repRNA. The engineered binding site may be designed and prepared such that it selectively binds to the predetermined enzyme. In some aspects of the invention, the predetermined enzyme is attached to a polypeptide that specifically binds to the engineered binding site—thus the binding of the attached polypeptide to the engineered binding site effectively attaches (which may also be referred to herein as “binds”) the predetermined enzyme to the engineered binding site.

A number of different engineered binding sites and corresponding binding partner molecule may be used in systems and methods of the invention. For example, though not intended to be limiting, an engineered binding site may be engineered MS2 binding site for which an MS2 Capsid protein (MCP) is its selective binding partner, and wherein the MCP is capable of binding to one of the MS2 binding sites that are engineered into the repRNA editing array. In certain aspects of the invention, an MCP protein may be attached to the predetermined enzyme and the binding of the MCP protein to its selective binding partner—the MS2 binding site—in the editing array of the repRNA molecule, thereby attaching the predetermined enzyme to the array. In certain aspects of the invention, the predetermined enzyme and the selective binding partner for the engineered binding site may be present as part of a fusion protein.

### Expression of Molecular Reporter Systems and Components

In certain aspects of the invention, a molecular reporter system or component thereof can be expressed in a cell and used to determine characteristics such as, but not limited to the timing of transcriptional events and the effect of stimuli on transcription in the cell. In some embodiments, a baseline determination of one or more characteristics of transcription in a “control” cell can be performed using a system of the invention. Such baseline determinations may be made for the same characteristics that are also determined in similar cells but under different circumstances. For example, a baseline determination may indicate a “control” characteristic which can be compared to the characteristic in a “test” cell that is exposed to one or more different stimuli, environmental changes, etc. to which the control cell was not exposed. For example, though not intended to be limiting, a test cell that includes a repRNA system of the invention can be contacted with a test agent such as a biological agent, etc. and a difference in one or more characteristics in the test cell compared to a control cell not contacted with the biological agent in order to ascertain whether there is an effect of the agent on transcription in the cell. Non-limiting examples of test agents are: a candidate compound, a pharmaceutical compound, an electrical stimulus, a chemical stimulus, a biological stimulus, a signaling molecule, a signaling chemical, a temperature stimulus, and a light stimulus. Additional stimuli and agents that are suitable for use in embodiments of the invention are known and routinely used in the art.

It will be understood that in some aspects of the invention, a stimulus or test agent may be delivered directly to a cell that includes a molecular recorder system of the invention, or may be delivered to another cell that is in communication with a cell that includes a molecular recorder of the invention. As used herein, the term “in communication with” used in reference to a cell that includes an RNA-based molecular recorder of the invention, includes cells, for example, that influence the cell comprising the RNA-based molecular recorder, for example, though not intended to be limiting, via a neurotransmitter means, an electrical means, etc. Communication can be direct communication from a cell immediately (directly) upstream from the cell that includes a molecular recorder system of the invention, or can be indirect communication, such as the result of activity of a cell further (indirectly) upstream that impacts the cell in which a molecular recorder of the invention is included. Stimulation of one or more of a cell directly upstream and a cell indirectly upstream may result in a change in transcription in a cell that includes a molecular recorder of the invention, and the presence of the molecular recorder permits determination of changes in characteristics of transcription in that cell using methods of the invention. As used herein a change in transcription means an alteration in the transcription characteristic, for example an increase in a rate or timing of transcription, a decrease in a rate or timing of transcription, the start of transcription, a delay in the start of transcription, and the like.

Methods and molecular recorder systems of the invention can be used to assess one or more changes in: (1) an internal environment of a cell, (2) an external environment of a cell, (3) an internal environment of an upstream cell, and (4) an external environment of an upstream cell. Non-limiting examples of events and situations that may change in a cell's internal or external environment and that can directly or indirectly effect transcription in a cell comprising a molecular recorder of the invention include, an action potential, a disease or injury condition in the cell or subject comprising the cell, contact of the cell with a test agent or compound, contact of the cell with a pharmaceutical agent or compound, a surgical procedure in the subject, contact of the cell with radiation, light, electric stimulation, etc. Other types of events and actions that alter the internal or external environment of a cell are known in the art, and can also be assessed using methods and RNA-based molecular recorders of the invention.

Components of RNA-based molecular recorder systems of the invention are well suited for targeting cells, expression in cells, and for use to detect and assess transcription levels and changes associated with stimuli and/or cell activities. In some embodiments, a molecular recorder system of the invention can be utilized to detect one or more of conductance changes across cell membranes, the impact of endogenous signaling pathways (such as calcium dependent signaling, etc.), and the effect of applied candidate compounds and agents on a cell that includes the molecular recorder of the invention. Thus, certain aspects of the invention include methods of using RNA-based molecular recorders to screen putative therapeutic agents, known therapeutic agents, combinations of two or more independently selected known and putative therapeutic agents. One or more RNA-based molecular recorders of the invention can also be used in some embodiments of methods of the invention to assess the effect of internal cellular conditions, environmental conditions external to the cell, and to assess the result diseases, injuries, treatments, etc. on transcription in the cell comprising the molecular recorder. Methods and systems of the invention can also be used to examine normal cells in vitro and in vivo. For example, in some embodiments a RNA-based molecular recorder system can be used to determine transcription events in normal cells and subjects and the resulting information on transcription characteristics can be applied in the study of normal cell development, non-limiting examples of which are cell development in regeneration, embryonic cell development, establishment of cell connectivity, and the like.

### Molecules and Compounds

The present invention, in part, includes novel RNA-based molecular recorder systems and components thereof, their expression in cells, and their use to determine alterations in characteristics of transcription in the host cell. As used herein, the term “host cell” means a cell that includes one or more components of an RNA-based molecular recorder system of the invention. Non-limiting examples of components of molecular recorder systems of the invention are described herein, see for example, Tables 1-3 and the Examples section. Aspects of the invention also include additional functional variants of components of RNA-based molecular recorder systems described herein, including polynucleotides, polypeptides, compositions comprising the components and functional variants thereof, and methods of using the components and functional variants thereof to perform RNA-based molecular recording in a cell., or a plurality of cells. As used herein the term “plurality of cells” means more than one cell, which in some embodiments of the invention is more than 1, more than 10, more than 100, more than 1000, more than 10,000, or more than 100,000, and more than 1,000,000, including all integers within the range from more 1 to more than 1,000,000

It is understood that the terms: RNA-based molecular recorder system components and tickertape system components encompass molecules, polypeptides, and polynucleotides described herein, as well as functional variants thereof. The invention also includes compounds and compositions that comprise one or more components of an RNA-based molecular recorder system of the invention. A compound or composition that comprises a component of a molecular recorder of the invention such as a predetermined enzyme or a repRNA may include only that component, may include both of those components, or may include one, two, three, four, five, six, or more additional elements. Non-limiting examples of additional elements are: a vector, a promoter, a detectable label sequence, a trafficking sequence, a delivery molecule sequence, an additional sequence, etc. The term “RNA-based molecular recorder” is used herein in reference to a repRNA and predetermined enzyme components or encoding molecules.

Certain embodiments of the invention include polynucleotides comprising nucleic acid sequences that encode a component of a molecular recorder system of the invention, and some aspects of the invention comprise methods of delivering and/or using such polynucleotides in cells, tissues, and/or organisms. RNA-based molecular recorder component polynucleotide sequences and amino acid sequences used in aspects and methods of the invention may be “isolated” sequences. As used herein, the term “isolated” used in reference to a polynucleotide, nucleic acid sequence, polypeptide, or amino acid sequence means a polynucleotide, nucleic acid sequence, polypeptide, or amino acid sequence, respectively, that is separate from its native environment and present in sufficient quantity to permit its identification or use. Thus, a nucleic acid or amino acid sequence that makes up a component of an RNA-based molecular recorder molecule that is present in one or more of a vector, a cell, a tissue, an organism, etc., may be considered to be an isolated sequence if it is not naturally present in that cell, tissue, or organism, and/or did not originate in that cell, tissue, or organism.

A host cell means a cell that comprises one or more components of an RNA-based molecular recorder. In certain aspects of the invention one or more components of an RNA-based molecular recorder system of the invention are delivered into and/or expressed in a cell. Examples of a host cells include, but are not limited to vertebrate cells, mammalian cells (including but not limited to non-human primate, human, dog, cat, horse, mouse, rat, etc.), insect cells (including but not limited to Drosophila, etc.), fish, worm, nematode, and avian cells. In some embodiments of the invention a cell is a plant cell.

One or more components of an RNA-based molecular reporter system of the invention may be derived from (also referred to herein as “being a variant of”) one or more components disclosed herein, and they may exhibit the same qualitative function and/or characteristics of the molecular reporter system component from which they have been derived, and/or may show one or more increased or decreased level of a function or characteristic of the parent component. In some embodiments of the invention an effectiveness of a variant or derived component of a molecular reporter system set forth herein may differ from the parent component. For example, in some instances a variant or derived component is capable of faster determination of a characteristic of transcription in a host cell than is possible for its parent component.

It is understood in the art that the codon systems in different organisms can be slightly different, and that therefore where the expression of a given protein from a given organism is desired, the nucleic acid sequence can be modified for expression within that organism. Thus, in some embodiments, a polynucleotide that encodes a component of an RNA-based molecular recorder system of the invention comprises a mammalian-codon-optimized nucleic acid sequence, which may in some embodiments be a human-codon optimized nucleic acid sequence. Codon-optimized sequences can be prepared using routine methods.

### Delivery of RNA-Based Molecular Recorder Components

Delivery of one or more components of an RNA-based molecular recorder of the invention to a cell and/or expression of the component in a cell can be done using art-known delivery means. [see for example, Chow et al. Nature 2010 Jan. 7;463(7277):98-102; and for Adeno-associated virus injection: Betley, J. N. & Sternson, S. M. (2011) Hum. Gene Ther. 22, 669-677; for In utero electroporation: Saito, T. & Nakatsuji, N. (2001) Dev. Biol. 240, 237-46; for microinjection into zebrafish embryos: Rosen J. N. et al., (2009) J. Vis. Exp. (25), e1115, doi:10.3791/1115; and for DNA transfection for neuronal culture: Zeitelhofer, M. et al., (2007) Nature Protocols 2, 1692-1704, the content of each of which is incorporated by reference herein in its entirety].

In some embodiments of the invention a component of an RNA-based molecular recorder of the invention is included as part of a fusion protein. It is well known in the art how to encode, prepare, and utilize fusion proteins that comprise a polypeptide sequence. In certain embodiments of the invention, a vector that encodes a fusion protein can be prepared and used to deliver a component of an RNA-based molecular recorder system of the invention to a cell and can also in some embodiments be used to target delivery of a component of an RNA-based molecular recorder system of the invention to a specific cell, cell type, tissue, or region in a subject. Suitable targeting sequences useful to deliver a component of an RNA-based molecular recorder of the invention to a cell, tissue, region of interest are known in the art. Delivery of a component of an RNA-based molecular recorder system of the invention to a cell, tissue, or region in a subject can be performed using art-known procedures. A fusion protein of the invention can be delivered to a cell by delivery of a vector encoding a fusion protein. The delivered fusion protein is then expressed in a specific cell type, tissue type, organ type, and/or region in a subject, or in vitro, for example in culture, in a slice preparation, etc.

In certain aspects of the invention, a component of an RNA-based molecular recorder system of the invention is non-toxic or substantially non-toxic to the cell into which it is delivered and/or expressed. In some embodiments of the invention, a component of an RNA-based molecular recorder of the invention is genetically introduced into a cell, and reagents and methods are provided for genetically targeted expression of components of an RNA-based molecular recorder system of the invention. Genetic targeting can be used to deliver one or more components of an RNA-based molecular recorder system of the invention to specific cell types, to specific cell subtypes, to specific spatial regions within an organism. In some embodiments of the invention, targeting can be used to control of the amount of a component of an RNA-based molecular recorder system of the invention that is expressed and the timing of the expression. Preparation, delivery, and use of a fusion protein and its encoding nucleic acid sequences are well known in the art. Routine methods can be used in conjunction with teaching herein to express one or more RNA-based molecular recorder system components and optionally additional polypeptides, in a desired cell, tissue, or region in vitro or in a subject.

### Vectors, Plasmids, and Molecules

Some embodiments of the invention include a reagent for genetically targeted expression of a component of an RNA-based molecular recorder system of the invention, wherein the reagent comprises a vector that contains the gene for the component. As used herein, the term “vector” refers to a nucleic acid molecule capable of transporting between different genetic environments another nucleic acid to which it has been operatively linked. The term “vector” may also refer to a virus or organism that is capable of transporting the nucleic acid molecule. One type of vector is an episome, i.e., a nucleic acid molecule capable of extra-chromosomal replication. Some useful vectors are those capable of autonomous replication and/or expression of nucleic acids to which they are linked. Vectors capable of directing the expression of genes to which they are operatively linked are referred to herein as “expression vectors.” Other useful vectors, include, but are not limited to viruses such as lentiviruses, retroviruses, adenoviruses, and phages. Vectors useful in some methods of the invention can genetically insert an RNA-based molecular recorder system of the invention into dividing and non-dividing cells and can insert an RNA-based molecular recorder system of the invention into an in vivo, in vitro, or ex vivo cell.

Vectors useful in methods of the invention may include additional sequences including, but not limited to one or more signal sequences and/or promoter sequences, or a combination thereof. Expression vectors and methods of their use are well known in the art. Non-limiting examples of suitable expression vectors and methods for their use are provided herein. In certain embodiments of the invention, a vector may be a lentivirus comprising the gene for an RNA-based molecular recorder system of the invention. A lentivirus is a non-limiting example of a vector that may be used to create stable cell line. The term “cell line” as used herein is an established cell culture that will continue to proliferate given the appropriate medium.

Promoters that may be used in methods and vectors of the invention include, but are not limited to, cell-specific promoters or general promoters. Methods for selecting and using cell-specific promoters and general promoters are well known in the art. A non-limiting example of a general purpose promoter that allows expression of an RNA-based molecular recorder system of the invention in a wide variety of cell types—thus a promoter for a gene that is widely expressed in a variety of cell types, for example a “housekeeping gene” can be used to express RNA-based molecular recorder system component(s) of the invention in a variety of cell types. Non-limiting examples of general promoters are provided elsewhere herein and suitable alternative promoters are well known in the art. In certain embodiments of the invention, a promoter may be an inducible promoter, examples of which include, but are not limited to tetracycline-on or tetracycline-off, or tamoxifen-inducible Cre-ER.

In some embodiments of the invention a reagent for expression of a component of an RNA-based molecular recorder system of the invention is a vector that comprises a gene encoding the component, and optionally a gene encoding one or more additional polypeptides. Vectors useful in methods of the invention may include additional sequences including, but not limited to, one or more signal sequences and/or promoter sequences, or a combination thereof. In certain embodiments of the invention, a vector may be a lentivirus, adenovirus, adeno-associated virus, or other vector that comprises a gene encoding RNA-based molecular recorder system component(s) of the invention. An adeno-associated virus (AAV) such as AAV8, AAV1, AAV2, AAV4, AAV5, AAV9, are non-limiting examples of vectors that may be used to express a fusion protein of the invention in a cell and/or subject. Expression vectors and methods of their preparation and use are well known in the art. Non-limiting examples of suitable expression vectors and methods for their use are provided herein. Other vectors that may be used in certain embodiments of the invention are provided in the Examples section herein.

Promoters that may be used in methods and vectors of the invention include, but are not limited to, cell-specific promoters or general promoters. A non-limiting examples promoters that can be used in vectors of the invention are: ubiquitous promoters, such as, but not limited to: CMV, CAG, CBA, and EF1a promoters; and tissue-specific promoters, such as but not limited to: Synapsin, CamKIIa, GFAP, RPE, ALB, TBG, MBP, MCK, TNT, and aMHC promoters. Methods to select and use ubiquitous promoters and tissue-specific promoters are well known in the art. A non-limiting example of a tissue-specific promoter that can be used to express a component of an RNA-based molecular recorder system of the invention in a cell such as a neuron is a synapsin promoter, which can be used to express the component in certain embodiments of methods of the invention. Additional tissue-specific promoters and general promoters are well known in the art and, in addition to those provided herein, may be suitable for use in compositions and methods of the invention. Other non-limiting examples of promoters that may be used in certain embodiments of methods of the invention are provided in the Examples section.

Additional molecules that can be administered and delivered to a cell in a method or system of the invention, include, but are not limited to: opsin polypeptides, detectable label polypeptides, fluorescent polypeptides, additional trafficking polypeptides, etc.

Non-limiting examples of detectable label polypeptides that may be included in a composition comprising a component of an RNA-based molecular recorder system of the invention are: green fluorescent protein (GFP); enhanced green fluorescent protein (EGFP), red fluorescent protein (RFP); yellow fluorescent protein (YFP), dtTomato, mCardinal, mCherry, DsRed, cyan fluorescent protein (CFP); far red fluorescent proteins, etc. Numerous fluorescent proteins and their encoding nucleic acid sequences are known in the art and routine methods can be used to include such sequences in fusion proteins and vectors, respectively, of the invention.

Additional sequences that may be included in a fusion protein comprising a component of an RNA-based molecular recorder system of the invention are trafficking sequences, including, but not limited to: Kir2.1 sequences and functional variants thereof, KGC sequences, ER2 sequences, etc. Trafficking polypeptides and their encoding nucleic acid sequences are known in the art and routine methods can be used to include and use such sequences in fusion proteins and vectors, respectively, of the invention.

Table 3 provides a list of plasmids that have been prepared and used in RNA-based molecular recorder systems and components of the invention. Addition plasmids may also be used, for example pCMV Tet3G (Clontech) has also been used in embodiments of the invention. Those skilled in the art will be able to prepare additional suitable plasmids using routine methods in conjunction with information provided herein.

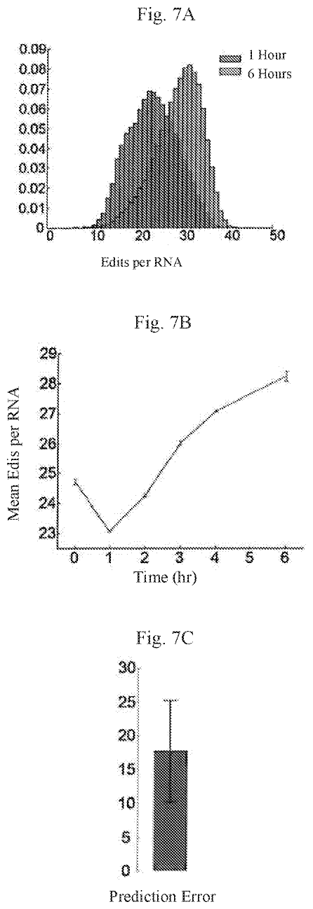

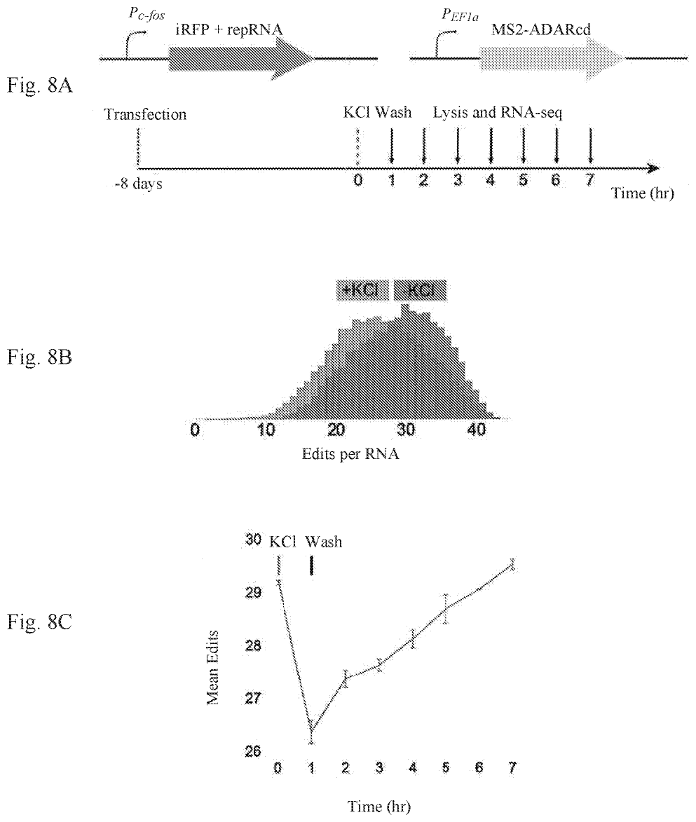

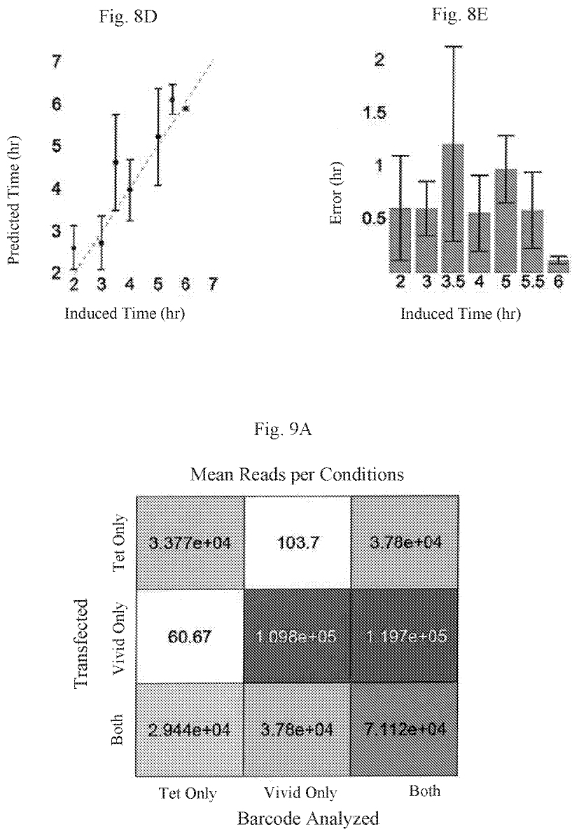

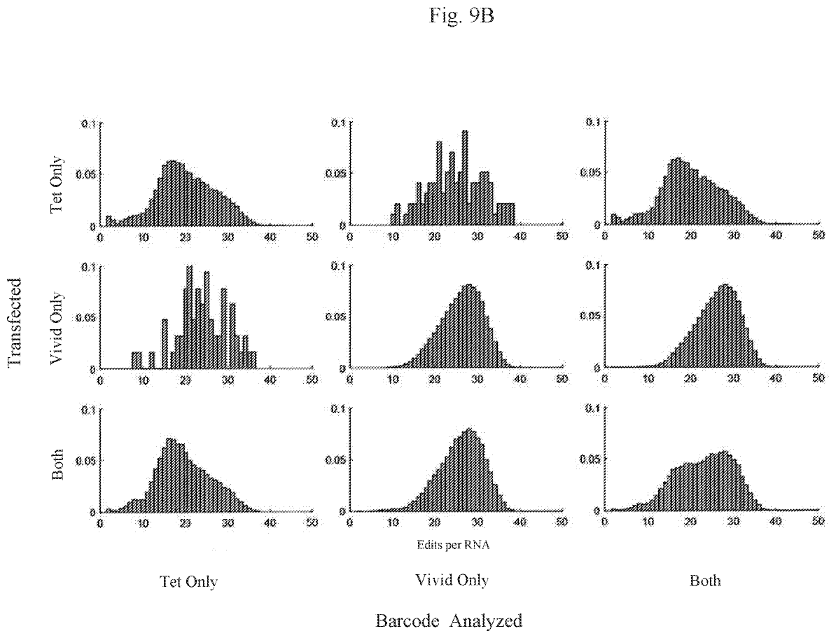

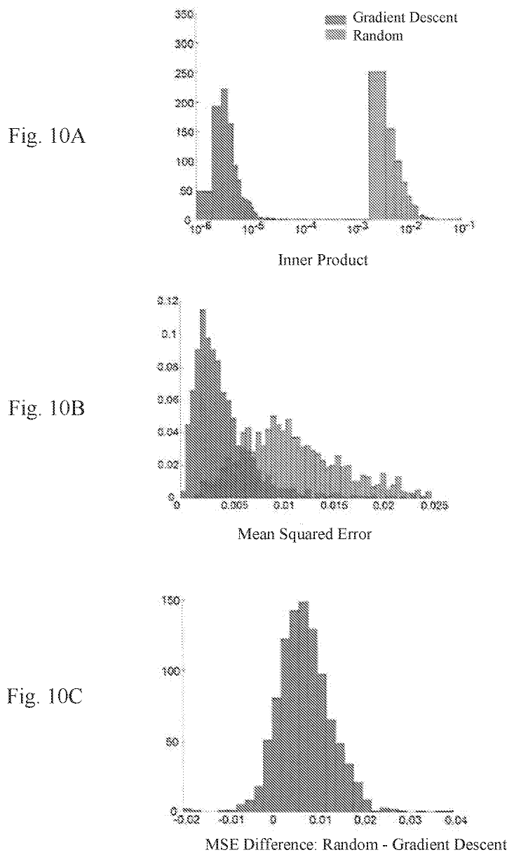

TABLE 3
Examples of plasmids used in repRNA systems and methods of the invention.

| Num | Name | Description | Used in |
|---|---|---|---|
| 116v1 | pAAV-Ef1a-MCP- | Fusion of MS2 coat protein to Drosophila ADAR E488Q, | FIG. 2B, |
| | dmADARE488Q | under Ef1a promoter, with WPRE | C |
| 116v5 | pAAV-Ef1a-MCP- | As with 116v1, but Human ADAR2 E488QT490A | All |
| | huADARE488QT490A | | plasmids |
| 116v6 | pAAV-Ef1a-MCP- | As with 116v1, but Human ADAR2 T490A | FIG. 2B, |
| | huADART490A | | C |
| 133 | pcDNA3.1-GAVPO | GAVPO (VIVID transactivator) expressed under the CMV | FIG. 7, |
| | | promoter in the pcDNA3.1 backbone. | Fig. 9 |
| 147B1 | pTRE3G-iRFP-B1- | repRNA Template A inserted into the 3′ UTR of iRFP | FIGS. 1, |
| | repRNA_A | between a bActin Zipcode element and a WPRE element, | 2, 5, 6, 9. |
| | | in the pTRE3G backbone, with RNA barcode TGC. Also | |
| | | includes a xrRNA element in the 5′ UTR. | |
| 148B1 | pTRE3G-iRFP-B1- | Same as 147B1, but with RNA Template B. | FIG. 2 |
| | repRNA_B | | |
| 149B1 | pLenti-5xUASG-iRFP-B1- | repRNA Template A inserted into the 3′ UTR of iRFP | FIG. 7 |
| | repRNA-A | between a bActin Zipcode element and a WPRE element, | |
| | | in a second generation lentiviral backbone with the Vivid | |
| | | promoter, with RNA barcode TGC. Also includes a xrRNA | |
| | | element in the 5′ UTR. | |
| 149B3 | pLenti-5xUASG-iRFP-B3- | Same as 149B1, but with RNA barcode CTG. | FIG. 9 |
| | repRNA-A | | |
| 187 | pTRE3G-c-fos-iRFP-B3- | Same as 147B1, with the TRE promoter removed and | FIG. 8 |
| | repRNA-A | replaced with a c-Fos promoter from pAAV-cFos-EYFP | |
| | | (Addgene 47907), and with RNA barcode CTG. | |

### Cells and Subjects

Some aspects of the invention include cells used in conjunction with an RNA-based molecular recorder system of the invention. Cells in which an RNA-based molecular recorder system component may be expressed, and that can be used in methods of the invention, include prokaryotic and eukaryotic cells. Certain embodiments of the invention, include use of mammalian cells; including but not limited to cells of humans, non-human primates, dogs, cats, horses, rodents, etc. In some embodiments of the invention, cells that are used are non-mammalian cells; including but not limited to insect cells, avian cells, fish cells, plant cells, etc. An RNA-based molecular recorder system of the invention may be included in non-excitable cells and in excitable cells, the latter of which include cells able to produce and respond to electrical signals. Examples of excitable cell types include, but are not limited, to neurons, muscle cells, visual system cells, sensory cells, auditory cells, cardiac cells, and secretory cells (such as pancreatic cells, adrenal medulla cells, pituitary cells, etc.), cardiac cells, immune system cells, etc.

Cells in which an RNA-based molecular recorder system of the invention can be used include embryonic cells, stem cells, pluripotent cells, mature cells, geriatric cells, as well as cells in other developmental stages. Non-limiting examples of cells that may be used in methods of the invention include: neuronal cells, nervous system cells, cardiac cells, circulatory system cells, kidney cells, liver cells, epiderminal cells, visual system cells, auditory system cells, secretory cells, endocrine cells, and muscle cells.

In some embodiments, a cell used in conjunction with methods and an RNA-based molecular recorder system of the invention is a healthy normal cell that is not known or suspected of having a disease, disorder, or abnormal condition. In some embodiments of the invention, a cell used in conjunction with methods and an RNA-based molecular recorder system of the invention may in some embodiments be a normal cell or in some embodiments is an abnormal cell. Non limiting examples of elements of an abnormal cell are: (1) a cell that has a disorder, disease, or condition; (2) a cell obtained from a subject that has, had, or is suspected of having disorder, disease, or condition; (3) a cell known to be or suspected of being involved in a disorder, disease, or condition; and (4) a cell that is a model for a disorder, disease, or condition, etc. Non-limiting examples of such cells are: a degenerative cell, a neurological disease-bearing cell, a cell model of a disease or condition, an injured cell, a cell downstream from a disease-bearing or injured cell, etc. In some embodiments of the invention, a cell may be a control cell. A cell that is directly or indirectly upstream from a cell in which an RNA-based molecular recorder system may be included may be a normal cell or may be an abnormal cell.

An embodiment of an RNA-based molecular recorder system of the invention may be included in a cell from or in culture, a cell in solution, a cell obtained from a subject, and/or a cell in a subject (in vivo cell). In some embodiments of the invention, an RNA-based molecular recorder system is present in and monitored in cultured cells, cultured tissues (e.g., brain slice preparations, etc.), and in living subjects, etc. As used herein, a the term “subject” may refer to a human, non-human primate, cow, horse, pig, sheep, goat, dog, cat, bird, rodent, fish, insect, or other vertebrate or invertebrate organism. In certain embodiments, a subject is a mammal and in certain embodiments a subject is a human. Additional non-limiting examples of cell types that may be used in certain methods of the invention are provided in the Examples section, as are non-limiting examples of organisms that may subjected to certain methods of the invention.

A cell that includes an RNA-based molecular recorder system and/or component of the invention may be a single cell, an isolated cell, a cell in culture, an in vitro cell, an in vivo cell, an ex vivo cell, a cell in a tissue, a cell in a subject, a cell in an organ, a cell in a cultured tissue, a cell in a neural network, a cell in a brain slice, a neuron, a cell that is one of a plurality of cells, a cell that is one in a network of two or more interconnected cells, a cell in communication with another cell, a cell that is one of two or more cells that are in physical contact with each other, etc. It will be understood that methods of the invention can be carried out in a plurality of cells such that one or more cells comprises the RNA based molecular recorder system of the invention. Inclusion of a system of the invention in a plurality of cells permits monitoring and determining one or more alterations in the composition of a repRNA across the plurality of cells.

### Controls

An RNA-based molecular recorder system of the invention and methods of using such molecular recorder systems can be utilized to assess changes in cells, tissues, and subjects in which the system is included. Some embodiments of the invention include use of an RNA-based molecular recorder system of the invention to identify effects of candidate compounds on cells, tissues, and subjects. Results of testing cell transcription activity using an RNA-based molecular recorder of the invention can be advantageously compared to a control. In some embodiments of the invention an RNA-based molecular recorder system may be in a cell or cell population and used to test the effect of candidate compounds on the cell or population, respectively. A “test” cell, tissue, or organism may be a cell, tissue, or organism in which activity of an RNA-based molecular recorder system of the invention can be determined or assayed. Results obtained using assays and tests of a test cell, tissue, or organism may be compared with results obtained from the assays and tests performed in other test cells, tissues, or organisms or assays and tests performed in control cells, tissues, or organisms.

As used herein a control value may be a predetermined value, which can take a variety of forms. It can be a single cut-off value, such as a median or mean. It can be established based upon comparative groups, such as cells or tissues that include an RNA-based molecular recorder system of the invention that is under essentially the same conditions of test cells but are not contacted with a candidate compound. Another non-limiting example of a comparative group includes cells or tissues that have a disorder or condition and groups without the disorder or condition. Another non-limiting example of comparative group includes cells from a subject or subjects with a family history of a disease or condition and cells from a subject or subjects without such a family history. A predetermined value can be arranged, for example, where a tested population is divided equally (or unequally) into groups based on results of testing. Those skilled in the art are able to select appropriate control groups and values for use in comparative methods of the invention.

### Administration Means

Administration of a component of an RNA-based molecular recorder system of the invention may include, but is not limited to: administering to a cell or subject a composition that includes a vector comprising a polynucleotide sequence that encodes the component, administering to a cell or subject a composition comprising the component, and administering to a subject a cell in which the component is present. A composition of the invention optionally includes a carrier, which may be a pharmaceutically acceptable carrier.

A component of an RNA-based molecular recorder system of the invention may be administered to a cell and/or subject in a formulation, which may be administered in pharmaceutically acceptable solutions, which may routinely contain pharmaceutically acceptable concentrations of salt, buffering agents, preservatives, compatible carriers, adjuvants, and optionally additional ingredients. In some aspects, a pharmaceutical composition comprises one or more RNA-based molecular recorder system component(s) of the invention and a pharmaceutically-acceptable carrier. Pharmaceutically acceptable carriers are well known to the skilled artisan and may be selected and utilized using routine methods. As used herein, a pharmaceutically acceptable carrier means a non-toxic material that does not interfere with the effectiveness of the biological activity of the active ingredients. Pharmaceutically acceptable carriers may include diluents, fillers, salts, buffers, stabilizers, solubilizers, and other materials that are well-known in the art. Exemplary pharmaceutically acceptable carriers are described in U.S. Pat. No. 5,211,657 and others are known by those in the art.

The terms “delivery into” and “include” when used herein to describe an action that results in a component of an RNA-based molecular recorder system of the invention being present in a cell, are intended to encompass delivery of the component(s) into the cell (for example, though not intended to be limiting, in the form of a fusion protein), and delivery of a polynucleotide sequence that encodes the component and that is subsequently expressed in the cell. A component of an RNA-based molecular recorder system of the invention may be administered using art-known methods. The absolute amount to be delivered can be determined using routine methods. The delivery may be done in a single administration, a single or multiple deliveries, and if delivered into a subject may be based on individual subject parameters including age, physical condition, size, weight, and the stage of a disease or condition, test parameters to be followed, etc. These factors can be addressed with no more than routine experimentation.

Various modes of administration will be known to one of ordinary skill in the art that can be used to effectively deliver one or more components of an RNA-based molecular recorder system of the invention in a desired cell, tissue, cell of a subject, organ of a subject, or region of a subject. Methods for administering a composition comprising a component of an RNA-based molecular recorder system of the invention may include, but are not limited to: injection, microinjection, perfusion, electroporation, or other suitable means. The invention is not limited by the particular modes of administration disclosed herein and additional art-known delivery means may be suitable for administration of components of an RNA-based molecular recorder system of the invention.

Other protocols suitable for administration of one or more components that are part of an RNA-based molecular recorder system of the invention are known to those in the art. Embodiments of methods of the invention to administer a cell or vector to increase a level of a component of an RNA-based molecular recorder system of the invention in an animal other than a human; and administration and use of an RNA-based molecular recorder system of the invention for testing purposes or veterinary purposes, are substantially the same as described above. It will be understood by a skilled artisan that this invention is applicable to both human and animals.

### Assessment Methods

Disorders, conditions, and events that may be assessed using methods of the invention to include an RNA-based molecular recorder of the invention in a cell, tissue, and/or subject and to use the system to determine characteristics of transcription in the cell. Methods and systems of the invention may be used to assess early stage development, cell and tissue regeneration, cell communication, disease, etc. Diseases that may be examined using methods and systems of the invention include, but are not limited to: injury, brain damage, spinal cord injury, epilepsy, metabolic disorders, cardiac dysfunction, vision loss, blindness, deafness, hearing loss, and neurological conditions (e.g., Parkinson's disease, Alzheimer's disease, and seizure), degenerative neurological conditions, drug contact, toxins, etc. In some embodiments of the invention, a disorder or condition may be monitored by including an RNA-based molecular recorder system of the invention in at least one cell and monitoring characteristics of transcription in the cells using the molecular recorder system. In some embodiments of the invention, such methods can be used in methods such as, but not limited to, assessing therapeutic agents and treatments, assessing putative therapeutic agents and treatments, expanding understanding of connectivity between cells, and exploring transcription activity patterns in a cell or cells. An RNA-based molecular recorder system of the invention may be targeted to cells and used to monitor transcription changes in such cells.

The present invention in some aspects, includes one or more of preparing nucleic acid sequences that encode one or more components of an RNA-based molecular recorder system of the invention, expressing in cells one or more components of an RNA-based molecular recorder system encoded by the prepared nucleic acid sequences; activating a promoter in the RNA-based molecular recorder system that activations the repRNAs in the system, and monitoring changes in transcription in the cell by assessing changes in editing of the composition of the repRNA The ability to specifically, consistently, reproducibly, and sensitively monitor changes in the repRNA composition using methods such as sequencing and single cell sequencing has been demonstrated. The present invention enables monitoring of transcription changes in in vivo, ex vivo, and in vitro, and the RNA-based molecular recorder system and its use have broad-ranging applications for drug screening, disease assessment, treatment assessment, and research applications, some of which are describe herein.

### EXAMPLES

### Example 1

### Materials and Methods:

### Cloning:

All plasmids were constructed either using restriction cloning using restriction enzymes from New England Biosciences and the NEB Quick Ligation kit (M2200L), or using the In-Fusion HD cloning enzyme mix (Clontech, 638911). Plasmids were grown in E.Cloni 10G Chemically Competent Cells (Lucigen, 60107-1) and were verified by Sanger sequencing (Eton biosciences). All plasmids are deposited on Addgene.

Due to high repetition present in the RNA editing templates, inserts for plasmids 76, 147, 148, 149, and 187 (see Table 1) were ordered as sense and antisense ultramer oligonucleotides, which were annealed to each other prior to cloning. Plasmid 76 was cloned by inserting RNA templates (A_Short, B_Short, C, D, E) into the 3′ UTR of an iRFP transcript expressed under a UbC promoter in a second generation lentivirus backbone using SphI and Clal. Subsequently, this plasmid was modified by the addition of a flavivirus xrRNA in the 5′ UTR. Templates A_Short and B_Short were then extended by inserting another pair of annealed ultramers on the 5′ side of A_Short and B_Short using SphI and MluI. The resulting templates are designated A and B. To generate plasmids 147, 148, 149, and 183 (used in certain experiments herein), templates A and B were then moved into different backbones and different promoters by restriction cloning, or by Gibson assembly with PCR amplification of the repRNA template region. Template A is used throughout the Examples, and Template B is shown in FIG. 2 for comparison.

### RNA Purification, Library Preparation, and Sequencing

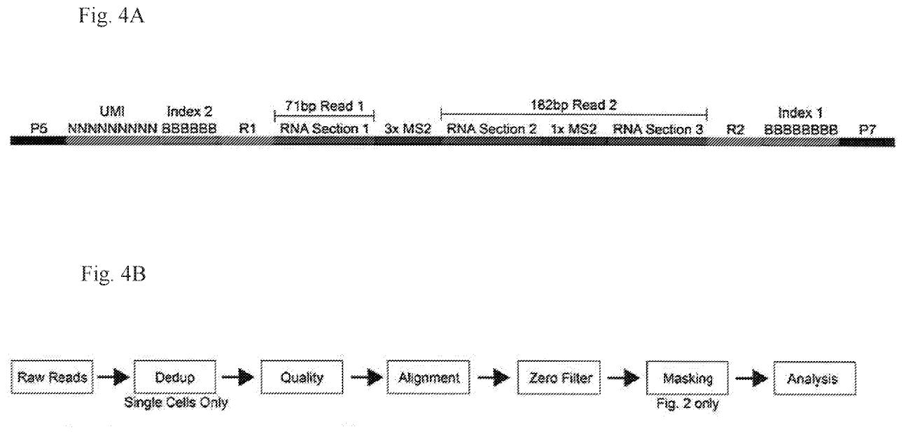

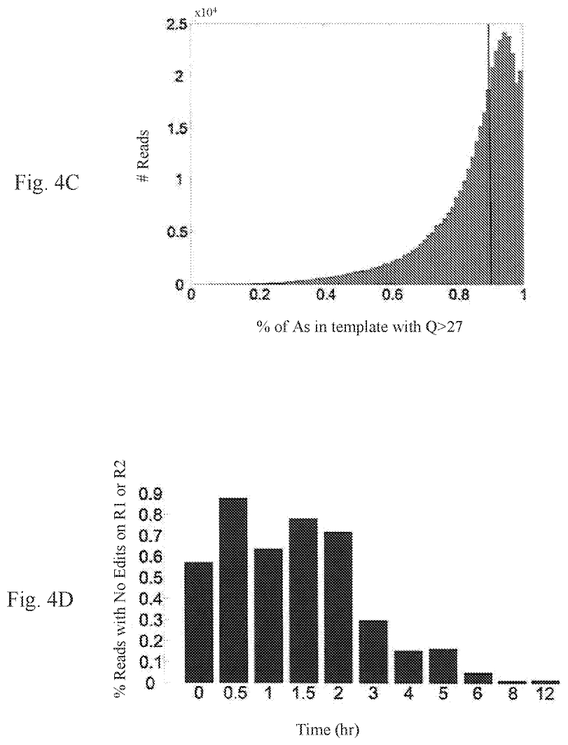

All cell cultures were lysed with 600μL of buffer RLT Plus from the Qiagen RNEasy Plus Mini Kit (Qiagen, 74136), and were pipetted up and down vigorously to homogenize. RNA was then purified using the Qiagen RNEasy Plus Mini kit, following the instructions from the manufacturer. Subsequently, 11 μL of purified RNA was reverse transcribed using Superscript IV (Thermofisher, 18090050) and a barcoded version of SGR-174 (see Table 1), following the protocol from the manufacturer. Reverse transcription reactions were then purified using Agencourt Ampure XP beads at a 1:1 dilution (Beckman-Coulter, A63881). Some portion of the eluent, typically 25%, was then PCRed using P5 and a barcoded version of SGR-176 (see Table 1) the Q5 Hot Start High Fidelity 2× Master Mix (NEB, M0492L) with the following settings: 30 s of 98° C. denaturation; then 25-30 cycles of 10 s denaturation at 98° C., 20 s annealing at 70° C., and then 25 s extension at 72° C. Neuron lysates were typically PCRed for 30 cycles, while HEK cell lysates were typically PCRed for 25 cycles. PCR reactions were then pooled and run on a gel, and a 400 bp band was extracted using the NucleoSpin PCR Cleanup Kit (Macherey-Nagel, 740609.250). The concentration of DNA in the resulting eluent was determined via a Qubit 2 fluorometer (Thermofisher), and was then adjusted to 4 nM for sequencing. The read structure is shown in FIG. 4A.

Sequencing was performed using NextSeq Mid Output 300 cycle kit (Illumina, FC-404-2004) or Miseq 300 cycle v2 kits (MS-102-2002), with at least 80 bp read 1 and 185 bp read 2, with 8 bp index 1 and 15 bp index 2.

### HEK and 3T3 Cell Culture:

Except in the case of the single cell experiments, HEK293FT and 3T3 cells were plated in 24 well plates. Cells were grown in DMEM (Thermofisher, 10566016), supplemented with Penicillin/Streptomycin (Thermofisher, 15140122) and 10% certified Tet-system approved FBS (Clontech, 631101). Transfections were performed using the TransIT-X2 system (Mirus, MIR 6000), following the manufacturer's instructions.

For doxycycline experiments, HEK and 3T3 cells in 24 well plates were transfected with 300 ng of plasmid 147 or 148, 100 ng of pCMV Tet3G from the Tet-on 3G system (Clontech, 631168), and 100 ng of plasmids 116v1, 116v5, or 116v6. In the experiments with results shown in FIGS. 1, 2, 5 and 6, they were transfected with both 147 and 148, and received 150 ng of each plasmid. At least 12 hours after transfection, cells were stimulated by adding doxycycline to a final concentration of 1 μg/mL, followed by gentle mixing or swirling of the plate. Subsequently, transcription was halted by adding Actinomycin D to a final concentration of 1 μg/mL in the same medium. After waiting for the experimental time period, cells were lysed using Buffer RLT Plus and libraries were prepared as described in the section above herein.

For experiments using the Vivid promoter, 3T3s were transfected with 300ng of plasmid 149, 100 ng of pCMV Tet3G, and 100 ng of plasmid 116v5. For conditions in which cells were transfected with both plasmid 147 and plasmid 149, they received 150 ng of each plasmid. For the experiments in FIG. 7, cells were stimulated with a blue LED (Thor Labs, M455L2) with a total power of 200 μW/cm². The LED was turned on for 1 hour, and was subsequently turned off. After the LED was turned off, the cells were wrapped in foil to prevent accidental light exposure. Cells were then lysed after the experimental time period.

### HEK Cell Doxycycline Experiment

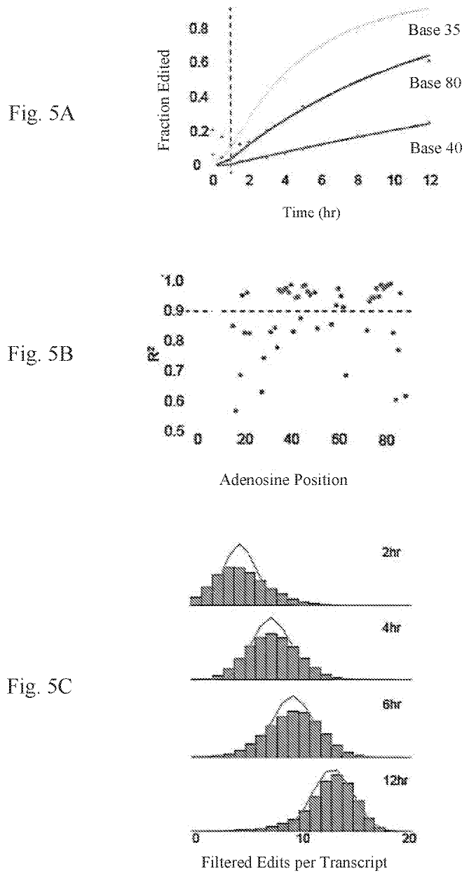

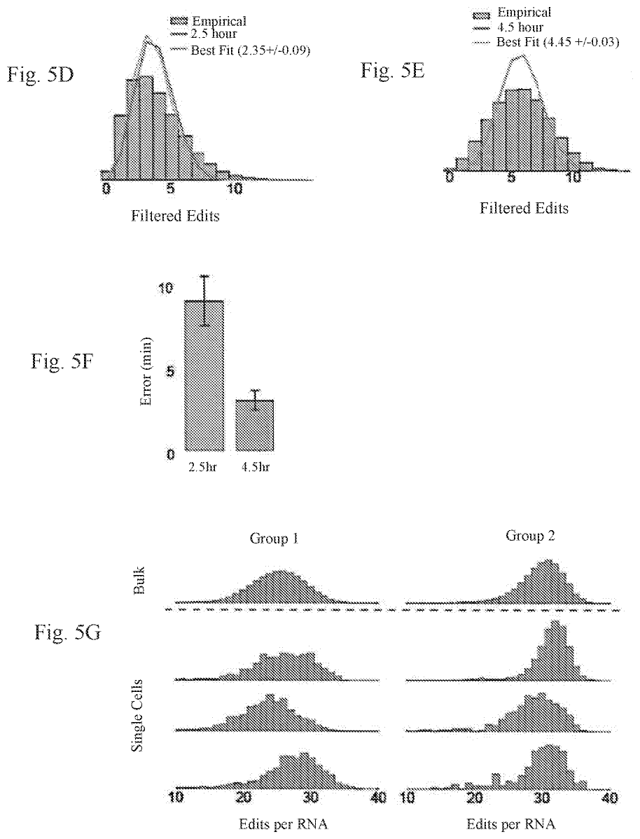

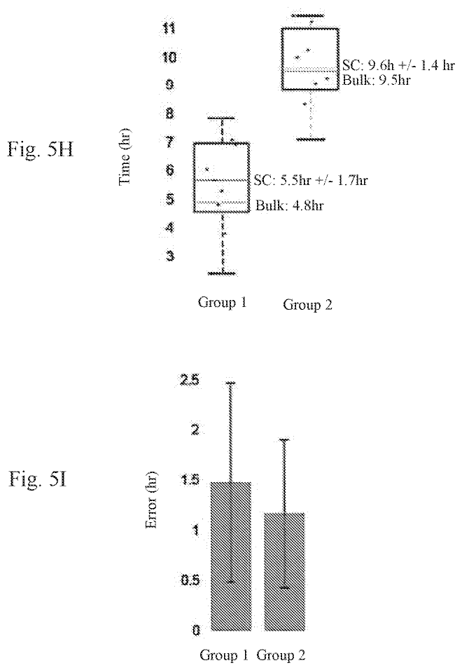

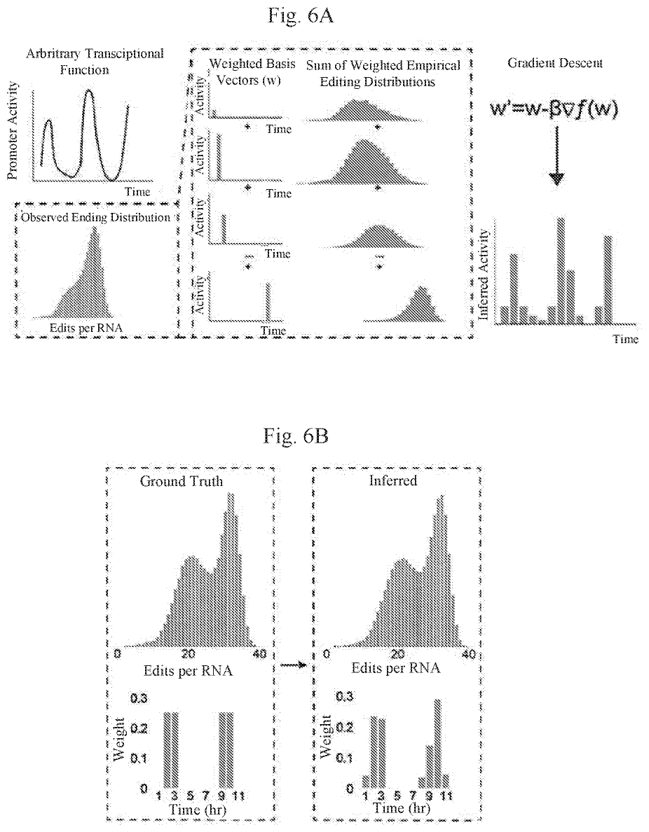

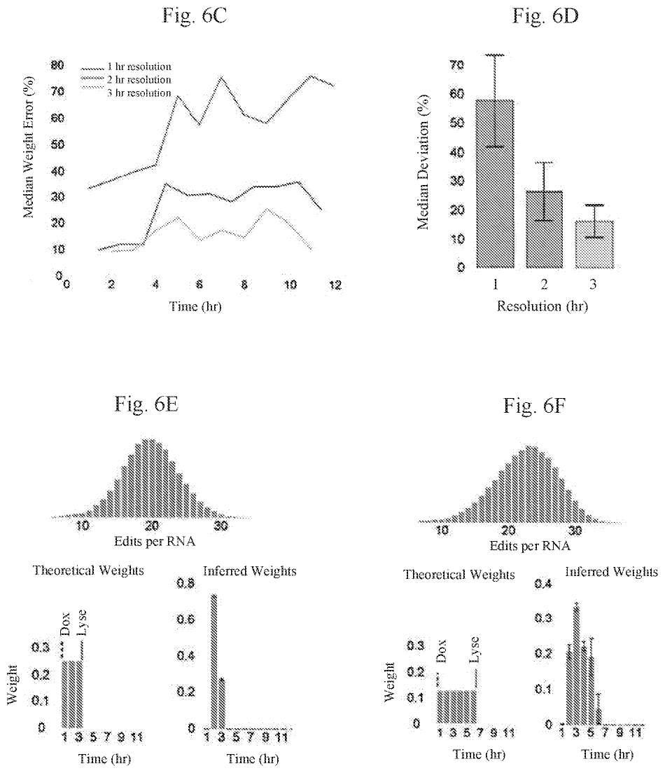

For the experiment with results shown in FIG. 1E-G and FIG. 5, cells were stimulated as described above and were lysed at the following time points: 0 hours (i.e., immediately before adding dox), 0.5 hours after adding dox, 1 hour after adding dox (i.e., immediately before adding ActD), 2 hours after adding dox, 3 hours after adding dox, 4 hours after adding dox, 5 hours after adding dox, 6 hours after adding dox, 8 hours after adding dox, and 12 hours after adding dox. Each time point consisted of three replicates. On a separate occasion, three replicates were collected at 2.5 hours after adding dox and 4.5 hours after adding dox, and these time points functioned as the test time points in FIGS. 5D,E. In preparation for the analysis for FIG. 6, time points were collected at 7 hours after adding dox, 9 hours after adding dox, 10 hours after adding dox, and 11 hours after adding dox.

### 3T3 Vivid Experiments:

For the experiment in FIG. 7, three replicates were collected for each of the following time points: immediately prior to turning on the LED, 1 hour after turning on the LED (i.e., immediately prior to turning off the LED), 2 hours after turning on the LED, 3 hours after turning on the LED, 4 hours after turning on the LED, and 5 hours after turning on the LED.

### Single Cell Experiments:

For all experiments involving single cells, HEK cell cultures were prepared, transfected with 100 ng of pAAV-CAG-GFP (Addgene 37825), 200 ng of plasmid 147, 100 ng of plasmid 116v5, and 100 ng of pCMV Tet3G, stimulated with doxycycline, and then silenced with actinomycin D as described above. Subsequently, at the designated time point (e.g., 8 hours or 4 hours after doxycycline was added to the culture medium), cells were treated with trypsin (Life Technologies, 25300054). Following trypsinization, cells were centrifuged at 850 g, washed in cold PBS, and then resuspended in cold PBS. 96 well plates were prepared, with each well containing a solution of 0.2% Triton-X with 2U/μL RNAse inhibitor. Individual cells were sorted into the wells of this wellplate using a Moflo Astrios EQ flow cytometer. Following sorting, the wellplate was sealed, centrifuged, and then placed at −80° C. overnight.

The single cell analysis was nominally conducted with cells from 4 hr and 8 hr time points. However, following trypsinization, cells remained in cold PBS for up to an hour and a half due to latencies in the sorting process. For this reason, estimates from the single cells were compared to the estimates for populations of ˜100,000 of the same cells (i.e., stored in cold PBS for the same amount of time) lysed immediately after sorting.

Library preparation for the single cells proceeded as follows. Plates containing single cells were thawed, and 7 μL of nuclease free water was added to the single cells to bring the total volume up to 11 μL. Subsequently, reverse transcription was performed using Superscript IV and the SGR-174 RT primers, as in the case of the bulk samples, with the following modifications. RT primers were distributed so that each cell at a given time point received an RT primer with a different barcode. In addition, for each time point, two no-template RT reactions were performed. Finally, after the 50° C. step in the Superscript IV protocol, the samples were cooled to 37° C. and 20U of Exonuclease 1 (NEB, M0293S) was added to the reaction to remove excess primers. Samples then remained at 37° C. for 10 minutes, before proceeding to the 80° C. heat inactivation step. Following reverse transcription, the RT reactions for all cells and the two no-template controls at a given time point were pooled, cleaned with Ampure XP beads at a 1:1 dilution, and were then PCRed using the same protocol as for the bulk samples. Cells were pooled prior to PCR as a way of reducing the number of cycles necessary to achieve amplification. In order to minimize barcode swapping between cells during the pooled PCR reaction, cells were excluded if they received fewer than 4 times the number of reads that the no-template controls received. In practice, this corresponded to a minimum of roughly 250 reads per cell.

### Neuron Culture Preparation and Transfection:

All procedures involving animals at MIT were conducted in accordance with the US National Institutes of Health Guide tier the Care and Use of Laboratory Animals arid approved by the ⁻Massachusetts Institute of Technology Committee on Animal Care. Primary hippocampal neuron culture was prepared as previously described. Neuron cultures were transfected at 6-7 DIV using a commercial calcium-phosphate kit (Thermofisher, K278001), as previously described. Briefly, neurons were transfected with 60Ong of pU⁻C19, 200ng of plasmid 116v5, and 200 ng of plasmid 187. Neurons were then incubated with calcium-phosphate precipitates for 30-60 minutes, followed by washing with MEM buffer at pH 6.7-6,8 to remove residual precipitates.

### Neuron Culture Stimulation:

Neurons were stimulated at 14-15DIV. Neurons were placed in 1 mL of plating medium (500 mL MEM, 2.5 g glucose, 50 mg transferrin, 1.1 g HEPES, 5 mL 200 mM L-Glutamine, 12.5 mg insulin, 50 mL HI FBS, 10 mL B27 supplement). To stimulate the neurons, 250 μL of 5× depolarization medium was added and the mixture was agitated gently. Neurons were then left for one hour in an incubator. Subsequently, the medium was aspirated and neurons were washed twice in plating medium. They were then left in plating medium for a variable amount of time, before being lysed in 600 μL of buffer RLT Plus.

### Plating Medium:

1. 500 mL MEM (Thermofisher, 51200-038)

2. 2.5 g glucose (Sigma Aldrich, G7528-1KG)

3. 50 mg transferrin (Sigma Aldrich, T1283-500 mg)

4. 1.1 g HEPES (Sigma Aldrich, H3375-500 G)

5. 5 mL 200mM L-Glutamine (Thermofisher, 25030-081)

6. 12.5 mg insulin (Millipore, 407709)

7. 50 mL HI FBS (VWR, 45000-736)

8. 10 mL B27 Supplement (Thermofisher, 17504-044)

### 5× Depolarization Medium

1. 170 mM KCl

2. 10 mM HEPES pH 7.4

3. 1 mM MgCl₂

4. 2 mM CaCl₂

### Neuron Inference Experiment:

Due to the limited availability of neuron culture at any given time, the data for FIG. 8 was conducted in two separate experiments, which can be considered to be biological replicates. The following time points were collected: prior to stimulation (i.e., immediately before adding depolarization medium); 1 hour after stimulation (i.e., immediately before washing the neurons in fresh medium); 2 hours after stimulation; 3 hours after stimulation; 3.5 hours after stimulation; 4 hours after stimulation; 5 hours after stimulation; 5.5 hours after stimulation; 6 hours after stimulation; 7 hours after stimulation.

The breakdown of the data in FIG. 8 by experiment is as follows. In the first experiment, two samples were collected prior to stimulation; three samples at 1 hour; three samples at 2 hours; three samples at 3 hours; three samples at 4 hours; and two samples at 5 hours. In the second experiment, one sample was collected at 2 hours, two samples at 3 hours, three samples at 3.5 hours, two samples at 4 hours, two samples at 5 hours, three samples at 5.5 hours, two samples at 6 hours, and two samples at seven hours.

### Multiplexing:

Experiments for FIG. 9 were conducted as follows. Three wells of 3T3 cells were transfected as described above with 10Ong each of pCMV Tet3G, plasmid 133, plasmid 147B1, plasmid 149B3, and plasmid 116v5. Three wells were transfected with 100 ng of pCMV Tet3G, 100 ng of plasmid 116v5, and 100 ng of plasmid 147B1, and 200 ng of pAAV-CAG-GFP. Finally, three wells were transfected with 100 ng of plasmid 133, 100 ng of plasmid 149B3, 100 ng of plasmid 116v5, and 200 ng of pAAV-CAG-GFP. Subsequently, all 9 wells were irradiated with blue light as described above for 1 hour, and were the placed in darkness. 7 hours after placing the cells in darkness, cells were stimulated with doxycycline as described above. After one hour in doxycycline, cells were lysed.

### Alignment and Edit Counting:

The alignment and analysis pipeline for sequencing data is summarized in FIG. 4B. Analysis of sequencing data was performed using custom Matlab code. Briefly, in the case of single cell data, de-duplication was first performed using a 9 bp UMI on the RT primer (oligo SGR-174). Other datasets were not de-duplicated. Reads were then filtered to ensure that they had the minimum necessary read length (67 bases on Read 1, and 184 bases on Read 2). Note that Read 1 was on the RT primer, so Read 1 reads the reverse complement of the RNA sequence. Thus, the expected mutation was A>G on Read 2, and T>C on Read 1. Alignment was performed using all bases that were not As on Read 2, or that were not Ts on Read 1. Reads were considered to be aligned to the template if 95% of the non-A (for Read 2) or non-T (for Read 1) bases matched the template. Furthermore, 90% of the bases that were expected to be As on Read 2 or Ts on Read 1 were required to have Q scores greater than 27 (FIG. 4C); reads that failed to achieve this threshold were discarded.

Finally, except as stated in FIG. 3, all reads were required to have at least one edit in Read 1 and at least one edit in Read 2 for analysis (FIG. 4D). This requirement was implemented because it appeared to eliminate a number of artifacts that were occasionally observed in the data: for example, each well would sometimes have different (large) numbers of RNAs with zero edits or one edit, which would confound attempts to infer timing from the mean editing rate, as in FIGS. 8 and 9. As a consequence of this requirement, all of the histograms of edits per RNA presented in the Examples herein appear not to show any RNAs with fewer than ˜12 edits. There are ˜12 bases in template A, all of which are on Read 2, that are edited much more quickly than any bases on Read 1. These are of the form UAG, and all form bulges in the RNA secondary structure, which is thought to encourage editing by ADAR. Exclusion of RNAs with zero edits on Read 1 or Read 2 limits the analysis to RNAs that are already fully edited at all 12 of those As, thus causing all RNAs to have at least 12 edits.

### Linear Interpolation:

In FIGS. 8 and 7, the time points associated with the c-fos neural activity and with the vivid promoter were determined by linear interpolation, as follows. First the mean number of edits per RNA were calculated for all replicates, and the mean across replicates was determined for each time point (plotted in FIG. 8B and 7B, designated Mₜ). Then, to perform the estimate, for each replicate R from time point t, the two time points t₁ and t₂ were identified such that t!=t₁, t₂ and such that the mean m_{R} of replicate R obeyed Mₜ₁<m_{R}<Mₜ₂. The time estimate for replicate R is then determined as

$${t}_{R} = \frac{{m}_{R} - {M}_{t 1}}{{M}_{t 2} - {M}_{t 1}} ( {t}_{2} - {t}_{1} ) + {t}_{1}$$

### Exponential Model:

The exponential model in FIG. 5 was implemented using custom code in Python, as follows. For each editable position i on the template, it was assumed that the likelihood of base i being edited followed an exponential distribution with parameter λᵢ, to be estimated from the data. Assuming an instantaneous pulse of transcriptional activity at time t=0, the fraction of edited bases for position i, yᵢ, could be modelled as the CDF of the exponential distribution:

yᵢ(t)=1−e^{-λ}^{ᵢ}^{t}

To more accurately capture the experimental setup, yi was modeled as an underlying process which is exponential, but with start time uniformly distributed in [0, tₛₜₒₚ], where t=0 represented when doxycycline was added to the cells and tₛₜₒₚ was the time at which actinomycin D was added to the cells. Specifically, a function of the form was fit

$${y}_{i} ( t ) = \{ \begin{matrix}1 - \frac{1 - {e}^{- {λ}_{i} t}}{{λ}_{i} t} if t \leq {t}_{stop} \\ 1 - \frac{{e}^{- {λ}_{i} ( t - {t}_{stop} )} - {e}^{- {λ}_{i} t}}{{λ}_{i} {t}_{stop}} if t > {t}_{stop}\end{matrix}$$

where tₛₜₒₚ was 1 hr and λᵢ was fit to the data using non-linear least squares. This function was fit for times t≥1.5 hr, because the editing distributions for earlier time points are strongly affected by populations of RNA present prior to doxycycline addition (for example, the mean editing rate in FIG. 1F decreases from t=0 to t=1). For the analysis in FIG. 5, analysis was performed using only those adenosines for which the R² of the resulting fit was greater than 0.9. The total number of edits was modeled to the RNA with a Poisson binomial distribution with N trials where N is the total number of editable positions and success probabilities given by yᵢ(t) for each position i. The probability of having n edits at time t is given by

$$p ( n , t ) = {\sum }_{A \text{:} s u m ( A ) = n} {\prod }_{k \text{:} {A}_{k} = 1}^{ } {y}_{k} ( t ) {\prod }_{j \text{:} {A}_{j} = 0} 1 - {y}_{j} ( t )$$

Here, A is a binary vector with each entry corresponding to a specific adenosine in the repRNA editing region. Aₖ=1 if adenosine k has been edited to inosine, and sum(A) counts the total number of edits in A. Time estimates using the exponential model were then made by minimizing the Kullback-Leibler divergence between p(n,t) and the empirical distribution q(n) over t. p(n,t) was calculated in practice via a dynamic programming approach.

For FIG. 5A-C, the exponential model was calculated using the data from a single replicate of the HEK doxycycline experiment. The distributions in FIG. 5C show the number of edits per RNA calculated across all bases with R² greater than 0.9 for that replicate, and the Poisson binomial model in FIG. 5C likewise included the same bases. By contrast, for FIG. 5D-I, bases were only retained if they had R² greater than 0.9 in all three replicates from the HEK doxycycline experiment. For this reason, the apparent numbers of edits per RNA are lower in FIG. 5D-I than in FIG. 5C.

### Gradient Descent:

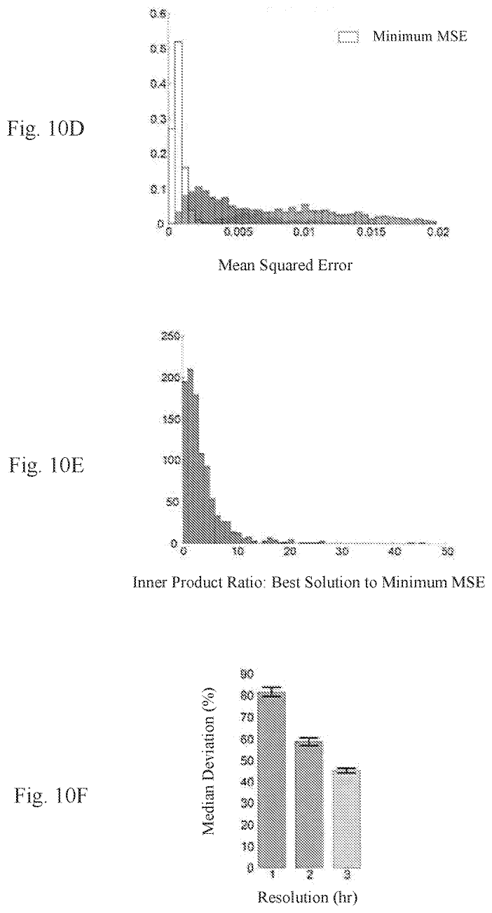

The gradient descent in FIG. 6 was implemented using custom code in Matlab. Briefly, the gradient descent algorithm was given an RNA editing distribution, which could either be an empirical distribution (FIG. 6E-F) or a simulated distribution (FIG. 6A-D). For FIG. 6A-D and 10, the simulated distributions were convex combinations of the editing histograms for a single replicate from the HEK doxycycline experiment. The gradient descent algorithm was also given a set of “basis vector” histograms, which were obtained by combining the data at each time point from all three replicates from the HEK doxycycline experiment. The gradient descent was then initialized by drawing a set of weights from a Dirichlet distribution with all parameters set to unity. The gradient descent minimized the mean squared error (L2 norm) between the input distribution and the convex combination of the basis vectors given by the weights. For each simulated distribution, the gradient descent was performed 1000 times and the solution was taken that minimized the L2 norm. For the analysis in FIG. 6 and FIG. 10, 1000 simulated distributions were generated from a Dirichlet distribution with all parameters set to unity.

Note that for the analysis in FIG. 6, additional time points were generated at 7 hr, 9 hr, 10 hr, and 11 hr using the same protocol used for experiments with results shown in FIGS. 1 and FIG. 5. However, those time points were not used in the analysis in FIG. 1 and FIG. 5.

### Results and Discussion

To assess the ability of an embodiment of an RNA-based molecular recorder system of the invention to report the timing of transcriptional activity, experiments were performed in which HEK293T cells expressing the RNA tickertape system were incubated under the control of the tetracycline response element (TRE) in medium containing doxycycline for one hour. Actinomycin D, which blocks RNA transcription by binding to DNA (FIG. 1D), was subsequently added. The results demonstrated that a population of unedited RNAs was generated following doxycycline induction, and that these RNAs became gradually more edited over time (FIG. 1E). Importantly, the repRNAs did not degrade following addition of actinomycin D (FIG. 3B). The very low variance observed between replicates indicated that this system could be used to infer the timing of the doxycycline pulse with high accuracy (FIG. 1F).

To assess and determine the timing of events in the TRE-tickertape system, a statistical model was designed that permits prediction of the RNA age distribution as a function of time since doxycycline induction. If the adenosines on the repRNA template are edited independently and uniformly in time, then for each adenosine on the repRNA, the fraction of RNAs with adenosines at that site should decrease exponentially with the time since transcription, with a site-specific rate constant that depends on the local sequence context. For each adenosine on the repRNA, an exponential cumulative distribution function (CDF) was fitted to the editing fraction over time at that base (FIG. 5A). Twenty four bases were identified that fit well to the model (i.e., for which the value of R² was greater than 0.9 across all replicates) (FIG. 5B). Analyzing only those bases, the distribution of edits per RNAs was well-approximated by a Poisson binomial distribution with a single parameter, t, which represents time since doxycycline was added to the medium (see Materials and Methods), with the weights in the Poisson binomial distribution given by the exponential CDFs (FIG. 5C). This Poisson binomial distribution was used to infer the times of cells induced at 2.5 and 4.5 hours prior to lysis, time points that had not been included in the dataset used to fit the exponential CDFs (FIG. 5D-E). By minimizing the Kullback-Leibler divergence (which is equivalent to maximizing the likelihood) between the test distributions and the Poisson binomial distribution overt, the timing of those events to was determined to be 2.35 hr±0.09 hr and 4.45 hr±0.03 hr (mean±s.d., N=3 technical replicates), respectively, indicating that tickertape can localize individual transcriptional events with 10 minute resolution time.

The Poisson binomial approach is a useful approach for estimation because it accounts for the exponential nonlinearity inherent in Poisson processes. However, it was also determined that a simple linear interpolation of the mean yielded accurate estimations in many cases. In the case of the TRE tickertape, the mean interpolation estimated the 2.5 hr and 4.5 hr time points as 2.53 hr±0.08 hr and 4.38 hr±0.02 hr (mean±s.d., N=3 replicates), with errors of 5 min±0.3 min and 7.5 min±1.1 min (mean±s.d., N=3 replicates), respectively.

To confirm that the accuracy of the RNA-based molecular recording system was not limited to the TRE tickertape or to HEK cells, similar experiments were performed in 3T3 cells using repRNAs expressed under a light-inducible Vivid promoter, induced with blue light for one hour (17, 18). The timing of light induction was estimated by interpolation of the mean number of edits per RNA, and yielded a temporal resolution of 17.7±7.5 minutes (FIG. 7, mean±s.d., N=9 samples total across three time points). The fact that tickertape works with multiple promoters supports the possibility of recording the activity of multiple promoters simultaneously in a single cell population, and this was validated using barcoded repRNAs responsive to the Tet and Vivid promoters (FIG. 9).

The accuracy of RNA tickertape depends on observing enough repRNAs that the empirical distribution of edits per repRNA accurately approximates the true distribution. Because individual cells may express thousands of copies of an mRNA, it was predicted that RNA tickertape is capable of accurate temporal predictions in single cells. Single HEK cells transfected with the TRE tickertape, induced with doxycycline, and then silenced with actinomycin D, were sorted into individual wells of a 96 well plate, followed by single-cell repRNA sequencing (FIG. 5F). The single cell estimates were unbiased, with the mean single cell estimates falling in both cases within one standard error of the bulk estimate (FIG. 5G). Moreover, the mean absolute errors observed were 1.46 hr±1.00 hr hours and 1.16 hr±0.74 hr for Group 1 and Group 2, respectively (mean±s.d., N=9 and N=10 cells, respectively), implying determination of timing of transcriptional events in single cells with ˜1 hour temporal resolution.

Having demonstrated the ability to detect the timing of one-hour transcriptional bursts, studies were performed to determine whether RNA tickertape is capable of decoding the time-course of arbitrary transcriptional programs, which was a much more challenging problem. It was determined that arbitrary transcriptional programs could be represented as convex weighted sums of the single-hour editing distributions (i.e., the one-hour “basis distributions”) as measured with the TRE tickertape (FIG. 6A). A gradient descent algorithm was built to minimize the L2 norm (i.e. summed squares of differences) between observed editing distributions and convex sums of these basis distributions. To evaluate the algorithm, simulated editing distributions resulting from arbitrary transcriptional programs were generated as convex weighted sums of the single-hour editing distributions measured in the TRE experiments. To avoid overfitting, the simulated distributions were comprised of convex sums of single-hour distributions from a single replicate, while the basis distributions used for fitting were comprised of the average distributions over all three replicates. Initial tests showed that the algorithm was able to faithfully reproduce complex patterns of transcriptional activity (FIG. 6B). In general, the gradient descent algorithm succeeded in reproducing the editing histogram with extraordinary accuracy (FIG. 10A). Additionally, the weight vector found by the gradient descent algorithm was on average much closer to the true weight vector than randomly sampled vectors (FIG. 10B), although this was not always true (FIG. 10C): because the simulated and approximated editing histograms were generated with different basis distributions, noise present in those basis distributions meant that the true weight vector was not in general the optimal solution for the gradient descent (FIG. 10D-E).

The gradient descent algorithm correctly approximated the weights of simulated distributions to within approximately 60% of the true values (FIG. 6C-D, green, 57.7%±15.8% mean±s.d., N=12 time points). Moreover, it was determined that temporal resolution could be traded for accuracy. Two-hour and three-hour running averages of the simulated weights were compared to two-hour and three-hour running averages of the approximated weights, and the results indicated that the deviations were 26.5%±10.1% (FIG. 6C-D, red, N=11 averages of 2 time points) and 16.3%±5.5% (FIG. 6C-D, yellow, N=10 averages of 3 time points), respectively. The deviations obtained in this way were significantly less than those obtained by comparing the same test distributions to random distributions (p<10⁻³ at 1 hr resolution; p<10⁻⁵ at 2 hr resolution; p<10⁻⁷ at 3 hr resolution, FIG. 10F). For many biological applications, transcriptional activity changes on the order of several hours, and studies have indicated that tickertape systems and methods of the invention can be used and are effective at recovering transcriptional programs on biologically relevant timescales.

Finally, to test whether the gradient descent algorithm is effective for decoding empirical editing distributions, studies were performed in which cells were stimulated with doxycycline for 3 or 6 hours, and applied the algorithm to the resulting empirical editing histograms (FIG. 6E-F). The resulting three- and six-hour empirical distributions were well-approximated by the gradient descent. The ground truth weights for these empirical systems are not known, making a quantitative assessment of accuracy impossible. However, in all three replicates, the gradient descent attributed the distributions to transcriptional activity lasting 2 consecutive hours in the first case, and 4-5 consecutive hours in the second case.

In certain experiments the repRNA expression was placed under the control of a c-fos promoter, and the tickertape system was transfected into primary mouse hippocampal neuron culture at 6 days in vitro (DIV), which is popular as a model for the study of coupling between excitation and transcription in neurons (20-21). At 14-15 DIV, neural activity was induced by adding a potassium-based depolarization medium to the culture (see Methods) (FIG. 8A). There was a clear shift in the repRNA editing histogram towards lower values following one hour of induction (FIG. 8B), indicating that new repRNAs were being produced in a depolarization-dependent manner.

To estimate the temporal history of neural activity, standards were generated by inducing neurons for one hour with the depolarization medium, which were washed back into normal (non-depolarizating) medium, and lysed at one hour intervals. For up to 7 hours after induction, a population of new repRNAs could be seen to gradually accumulate edits. Even in the presence of a large population of background repRNAs generated by constitutively fos+neurons, the mean number of edits per RNA increased linearly over time (FIG. 8C), at a rate of approximately 0.5 edits per hour. The linearity of the editing mean indicates that the editing mean should be a good predictor of the time elapsed since depolarization. As a first test, the times of each replicate were estimated for the 2 hr, 3 hr, 4 hr, 5 hr, and 6 hr time points by linear interpolation (see Methods). Results indicated that these replicates could be predicted from the standards with an average accuracy of 37±23 minutes (FIG. 8D-E, mean±s.d.). Then, as a follow-up study, neurons were we stimulated at 3.5 and 5.5 hour time points, and results indicated that these could be predicted with an average accuracy of 72±55 and 35±22 minutes, respectively. Thus, for most time points, tickertape permitted recording neural activity with approximately 30 minute time resolution.

A primary challenge in the detection of neural activity using tickertape is the presence of a large number of fos+ cells at baseline in primary hippocampal neuron culture. For this reason, tickertape applied to the readout of individual neuron activity, or to targeted populations of neurons outperforms the bulk measurements as described.

### REFERENCES

1. S. D. Perli et al., Continuous genetic recording with self-targeting CRISPR-Cas in human cells. Science. 353, 339-342 (2016).

2. F. Farzadfard, N. Gharaei, Y. Higashikuni, G. Jung, Single-Nucleotide-Resolution Computing and Memory in Living Cells. bioRxiv (2018).

3. F. Farzadfard, T. K. Lu, Genomically encoded analog memory with precise in vivo dna writing in living cell populations. Science. 346 (2014), doi:10.1126/science.1256272.

4. R. Kalhor et al., Rapidly evolving homing CRISPR barcodes. Nat. Methods. 14, 195-200 (2017).

5. R. U. Sheth, S. S. Yim, F. L. Wu, H. H. Wang, Multiplex recording of cellular events over time on CRISPR biological tape. Science. 358 (2017), doi:10.1126/science.aao0958.

6. W. Tang, D. R. Liu, Rewritable multi-event analog recording in bacterial and mammalian cells. Science. 360 (2018).

7. A. Shur, R. M. Murray, Proof of concept continuous event logging in living cells. bioRxiv (2018).

8. K. L. Frieda et al., Synthetic recording and in situ readout of lineage information in single cells. Nature. 541, 107-111 (2016).

9. S. L. Shipman et al., Molecular recordings by directed CRISPR spacer acquisition. Science. 353 (2016), doi:10.1126/science.aaf1175.

10. B. M. Zamft et al., Measuring cation dependent DNA polymerase fidelity landscapes by deep sequencing. PLoS One. 7 (2012), doi:10.1371/journal.pone.0043876.

11. D. Zenklusen, D. R. Larson, R. H. Singer, Single-RNA counting reveals alternative modes of gene expression in yeast. Nat. Struct. Mol. Biol. 15, 1263-1271 (2008).

12. K. D. Piatkevich et al., A robotic multidimensional directed evolution approach applied to fluorescent voltage reporters. Nat. Chem. Biol. 14 (2018), doi:10.1038/s41589-018-0004-9.

13. M. M. Matthews et al., Structures of human ADAR2 bound to dsRNA reveal base-flipping mechanism and basis for site selectivity. Nat. Struct. Mol. Biol. 23, 426-433 (2016).

14. A. Kuttan, B. L. Bass, Mechanistic insights into editing-site specificity of ADARs. Proc. Natl. Acad. Sci. 109, E3295-E3304 (2012).

15. T. Eifler, S. Pokharel, P. A. Beal, RNA-seq analysis identifies a novel set of editing substrates for human ADAR2 present in saccharomyces cerevisiae. Biochemistry. 52, 7857-7869 (2013).

16. E. Bertrand et al., Localization of ASH1 mRNA Particles in Living Yeast. Mol. Cell. 2, 437-445 (1998).

17. X. Wang, X. Chen, Y. Yang, Spatiotemporal control of gene expression by a light-switchable transgene system. Nat. Methods. 9, 266-271 (2012).

18. Z. Ma, Z. Du, X. Chen, X. Wang, Y. Yang, Fine tuning the LightOn light-switchable transgene expression system. Biochem. Biophys. Res. Commun. 440, 419-423 (2013).

19. A. H. Marblestone et al., Physical principles for scalable neural recording. Front. Comput. Neurosci. 7,137 (2013).

20. A. E. West et al., Calcium regulation of neuronal gene expression. Proc. Natl. Acad. Sci. U S. A. 98, 11024-31 (2001).

21. A. E. West, E. C. Griffith, M. E. Greenberg, Regulation of transcription factors by neuronal activity. Nat. Rev. Neurosci. 3, 921-931 (2002).

It is to be understood that the methods, compositions, and apparatus which have been described above are merely illustrative applications of the principles of the invention.

Numerous modifications may be made by those skilled in the art without departing from the scope of the invention. Although the invention has been described in detail for the purpose of illustration, it is understood that such detail is solely for that purpose and variations can be made by those skilled in the art without departing from the spirit and scope of the invention which is defined by the following claims. The contents of all references, patents and published patent applications cited throughout this application are incorporated herein by reference in their entirety.

We claim:

1. A molecular recording system, comprising a reporter RNA (repRNA) and a predetermined enzyme, wherein an alteration in an original composition of the repRNA indicates one or more of (a) an age of the repRNA and (b) a response of the repRNA to an applied stimulus.

2. The molecular recording system of claim 1, wherein the repRNA comprises an editing array in the 3′ UTR of a target RNA, wherein (i) the editing array comprises one or more engineered binding sites; and (ii) the editing array comprises one or more selectively favored substrates of the predetermined enzyme.

3. The molecular recording system of claim 2, wherein the target RNA is an endogenous RNA.

4. The molecular recording system of claim 2, wherein the target RNA is an mRNA.

5. (canceled)

6. The molecular recording system of claim 2, wherein the predetermined enzyme is attached to a binding polypeptide capable of binding to the engineered binding site in the repRNA editing array.

7. The molecular recording system of claim 2, wherein the engineered binding site is an engineered MS2 binding site.

8. The molecular recording system of claim 7, wherein the binding polypeptide is an MS2 Capsid protein (MCP) and is capable of binding the MS2 binding site engineered into the repRNA editing array.

9-14. (canceled)

15. The molecular recording system of claim 1, wherein the alteration in an original composition of the repRNA comprises a sequence edit in the repRNA sequence.

16. (canceled)

17. The molecular recording system of claim 2, wherein the editing array sequence is capable of accumulating sequence edits over time, and wherein determining the number of accumulated sequence edits in the editing array over a time period determines the age of the repRNA.

18. The molecular recording system of claim 2, wherein determining one or more edits in the editing array sequence over a time period indicates a repRNA response to the applied stimulus.

19. The molecular recording system of claim 18, wherein the number of edits in the editing array sequence corresponds to a temporal record of activation of a promoter that generates the repRNAs, and a number of edits in the repRNA corresponds to the length of time elapsed since the activation of the promoter.

20-21. (canceled)

22. The molecular recording system of claim 1, wherein the applied stimulus activates a promoter and activation of the promoter generates new repRNAs.

23. The molecular recording system of claim 2, wherein the target RNA encodes a detectable protein.

24. (canceled)

25. The molecular recording system of claim 1, wherein the alteration in the repRNA composition comprises a change in the repRNA sequence, wherein the change comprises one or more of: (i) a modification of one or more nucleic acids in the repRNA RNA sequence, and (ii) one or more of a substitution, deletion, and addition of a nucleic acid in the repRNA RNA sequence.

26-27. (canceled)

28. The molecular recording system of claim 1, wherein the determined repRNA composition is compared to a control repRNA composition and detection of one or more differences between the repRNA composition and the control repRNA composition indicates a change in the repRNA composition.

29-35. (canceled)

36. A cell comprising a molecular recording system comprising a reporter RNA (repRNA) and a predetermined enzyme, wherein an alteration in an original composition of the repRNA indicates one or more of (a) an age of the repRNA and (b) a response of the repRNA to an applied stimulus.

37-38. (canceled)

39. A vector comprising one or both of the repRNA and predetermined enzyme of claim 1.

40. A cell comprising the vector of claim 39.

41. (canceled)

42. A method of molecular recording in a cell, the method comprising including in a cell, a molecular recording system comprising a reporter RNA (repRNA) and a predetermined enzyme, wherein an alteration in an original composition of the repRNA indicates one or more of (a) an age of the repRNA and (b) a response of the repRNA to an applied stimulus.

43-46. (canceled)

47. The method of claim 42, further comprising determining an age of the RepRNA.

48-50. (canceled)
